(19) United States
(12) Patent Application Publication
(10) Pub. No.: US 2023/0026844 A1
(43) Pub. Date: Jan. 26, 2023

(54) Inhibitors of Cancer Biomarkers and Uses Thereof

(71) Applicants: Shengyun FANG, Silver Spring, MD (US); Yongwang ZHONG, Catonsville, MD (US)

(72) Inventors: Shengyun Fang, Silver Spring, MD (US); Yongwang Zhong, Catonsville, MD (US)

(73) Assignee: University of Maryland, Baltimore, Baltimore, MD (US)

(21) Appl. No.: 17/784,074

(22) PCT Filed: Dec. 12, 2020
(86) PCT No.: PCT/US2020/064720

§ 371 Date: Jun. 9, 2022

Related U.S. Application Data
(60) Provisional application No. 63/082,065, filed on Sep. 23, 2020.
(60) Provisional application No. 62/947,214, filed on Dec. 12, 2019.

(51) Int. Cl. C12N 15/113; A61P 35/00; A61K 45/06; A61K 31/713

(52) U.S. Cl. CPC C12N 15/113; A61P 35/00; A61K 45/06; A61K 31/713; C12N 2310/14

(57) ABSTRACT

Provided herein is an inhibitor for decreasing cellular levels X-box-binding protein 1 variant 1 (Xv1) in a cancer cell and a method for decreasing Xv1 in a cancer cell by contacting the cancer cell with this inhibitor. Also provided is a pharmaceutical composition for treating a cancer and a method for treating a cancer by administering this composition. In addition there is provided a kit for targeting Xv1 with at least one Xv1 inhibitor, at least one pharmaceutically acceptable carrier and a means for detecting the Xv1 protein or mRNA.

### CROSS-REFERENCE TO RELATED APPLICATIONS

This international application claims the benefit of priority under 35 U.S.C. § 119(e) of provisional application U.S. Ser. No. 63/082,065, filed Sep. 23, 2020, and provisional application U.S. Ser. No. 62/947,214, filed Dec. 12, 2019, both of which are hereby incorporated by reference in their entireties.

### BACKGROUND OF THE INVENTION

### Field of the Invention

The present invention relates to the fields of molecular biology and oncology. More particularly, the present invention relates to pharmaceutical compositions and methods for treating cancer by reducing levels or activities of X-box-binding protein 1 variant 1 (Xv1, ENST00000405219.7) in cancer cells.

### Description of the Related Art

Alternative start sites, alternative splicing, and alternative promoters are frequently used to regulate tissue or cancer-specific transcription (7,8). Tumors have up to 30% more alternative splicing events than in normal tissues (9). However, the translational status and functions of alternative transcripts in the tumors remain poorly understood (7,8,10). Nor is there information on whether reductions in expression levels of alternatively spliced proteins could treat cancers. Thus, there is a need in the art for methods of treating cancer by administering a pharmaceutical composition that reduce cellular levels of alternatively spliced proteins. The present invention fulfills this long-standing need and desire in the art.

### SUMMARY OF THE INVENTION

The present invention is directed to an inhibitor for decreasing cellular levels or activities of X-box binding protein 1 variant (Xv1) in a cancer cell.

The present invention is also directed to a method for decreasing a X-box binding protein 1 variant (Xv1) in a cancer cell comprising contacting the cancer cell with the above inhibitor.

The present invention is also directed to a pharmaceutical composition for treating a cancer. The pharmaceutical composition comprises X-box binding protein 1 variant (Xv1) inhibitor; and at least one pharmaceutically acceptable carrier.

The present invention is also directed method for treating a cancer in a subject by administering to the subject a pharmaceutically acceptable amount of the above pharmaceutical composition. The present invention is also directed to a related method further comprising, administering to the subject, at least one additional anti-cancer drug.

The present invention is further directed to a kit for targeting an X-box binding protein 1 variant (Xv1). The kit comprises at least one Xv1 inhibitor, at least one pharmaceutically acceptable carrier, a means for detecting the Xv1 protein or X-box protein mRNA and instructions for using the kit.

Other and further aspects, features, benefits, and advantages of the present invention will be apparent from the following description of the presently preferred embodiments of the invention given for the purpose of disclosure.

### BRIEF DESCRIPTION OF THE FIGURES

FIG. 1 is a schematic of X-box-binding protein 1 (XBP1) variant transcripts and proteins.

FIGS. 2A-2K show relative expression level of each XBP1 isoform in the transcriptomic datasets in Genotype-Tissue Expression (GTEX) and The Cancer Genome Atlas (TCGA). FIG. 2A shows relative expression level of each XBP1 isoform in all samples.

FIG. 2B shows relative expression level of each XBP1 isoform in lung adenocarcinoma (LUAD) and lung squamous cell cancer (LSCC). FIG. 2C shows relative expression level of each XBP1 isoform in prostate adenocarcinoma (PRAD). FIG. 2D shows relative expression level of each XBP1 isoform in colon adenocarcinoma (COAD). FIG. 2E shows relative expression level of each XBP1 isoform in testicular germ cell tumor (TGCT). FIG. 2F shows relative expression level of each XBP1 isoform in breast cancer (BRCA). FIG. 2G shows relative expression level of each XBP1 isoform in pancreatic cancer (PAAD). FIG. 2H shows relative expression level of each XBP1 isoform in liver hepatocellular carcinoma (LIHC). FIG. 2I shows relative expression level of each XBP1 isoform in sarcoma (SARC). FIG. 2J shows relative expression level of each XBP1 isoform in bladder urothelial carcinoma (BLCA). FIG. 2K shows relative expression level of each XBP1 isoform in head and neck squamous cell carcinoma (HNSC).

FIGS. 3A-3F show survival analysis using the Gene Expression Profiling Interactive Analysis (GEPIA2) tool. FIG. 3A shows survival analysis for breast cancer cells. FIG. 3B shows survival analysis for pancreatic cancer cells. FIG. 3C shows survival analysis for hepatocellular carcinoma cancer cells. FIG. 3D shows survival analysis for sarcoma cells.

FIG. 3E shows survival analysis for bladder urothelial cancer cells. FIG. 3F shows survival analysis for head and neck squamous cell carcinoma cells.

FIG. 4 shows quantitative RT-PCR analysis for stage-dependent expression of Xv1 in breast cancer tissues.

FIGS. 5A-5D show mRNA levels in various cancer cell lines. FIG. 5A is a schematic showing positions of the specific RT-PCR primers for XBP1, and Xv1. FIG. 5B show mRNA levels in the indicated cancer cell lines. FIG. 5C show mRNA levels in additional cancer cell lines. FIG. 5D show mRNA levels in cancer cell lines BT474 and MCF7ca and in non-cancerous cells HDFa (human primary dermal fibroblasts), GM05294 and MCF10A.

FIGS. 6A-6N show the effects of siRNA on susceptibility to apoptosis. FIG. 6A shows caspase 3/7 activity in BT474 cells incubated with negative control, Xv1 or XBP1 siRNAs.

FIG. 6B shows caspase 3/7 activity in HeLa cells incubated with negative control, Xv1 or XBP1 siRNAs. FIG. 6C shows mRNA levels of Xv1 and XBP1 following siRNA treatment in BT474 and HeLa cells. FIG. 6D shows caspase 3/7 activity in MCF10A cells incubated with negative control, Xv1 or XBP1 siRNAs. FIG. 6E shows caspase 3/7 activity in HDFa cells incubated with negative control, Xv1 or XBP1 siRNAs. FIG. 6F demonstrates the specificity and effectiveness of the siRNA knockdown in the MCF10A and HDFa cells. FIG. 6G shows representative immunoblotting analysis for cleaved caspase 3 and PARP-1 in BT474 and HeLa cells to confirm that knockdown of Xv1 but not XBP1 induced apoptosis in cancer cells.

FIG. 6H shows caspase 3/7 activity in MCF7ca cells incubated with negative control, Xv1 or XBP1 siRNAs. FIG. 6I shows caspase 3/7 activity in U251 cells incubated with negative control, Xv1 or XBP1 siRNAs. FIG. 6J shows caspase 3/7 activity in HepG2 cells incubated with negative control, Xv1 or XBP1 siRNAs. FIG. 6K shows caspase 3/7 activity in U2OS cells incubated with negative control, Xv1 or XBP1 siRNAs. FIG. 6L shows caspase 3/7 activity in GM05294 cells incubated with negative control, Xv1 or XBP1 siRNAs. FIG. 6M shows representative immunoblotting analysis for cleaved caspase 3 and PARP-1 in MCF7ca and U251 cells to confirm that knockdown of Xv1 but not XBP1 induced apoptosis in cancer cells. FIG. 6N shows representative images of cleaved caspase 3/7 substrates at day 4 after siRNA transfection.

FIGS. 7A-7D show the effect of Xv1 knockdown and re-expression on cell survival. FIG. 7A shows the effects of Xv1 and XBP1 knockdown on cell proliferation in BT474 cells. FIG. 7B shows the effects of Xv1 and XBP1 knockdown on cell proliferation in HeLa cells. FIG. 7C shows rescue of Xv1 depleted BT474 cells from cell death by re-expression of Xv1. FIG. 7D shows rescue of Xv1 depleted HeLa cells from cell death by re-expression of Xv1.

FIG. 8 shows the effect of Xv1 or XBP1 knockdown on anchorage-independent growth of BT474 cells.

FIGS. 9A-9E show the effect of Xv1 and XBP1 knockdown on in vivo tumor growth. FIG. 9A shows the effect of Xv1 and XBP1 knockdown on tumor volume of BT474 breast cancer in a mouse xenograft model. FIG. 9B shows the effect of Xv1 and XBP1 knockdown on tumor weight of BT474 breast cancer in a mouse xenograft model. FIG. 9C shows H&E staining for animals injected with BT474 breast cancer cells transfected with negative control siRNA. FIG. 9D shows H&E staining for animals injected with BT474 breast cancer cells transfected with Xv1 siRNA. FIG. 9E shows H&E staining for animals injected with BT474 breast cancer cells transfected with XBP1 siRNA.

FIGS. 10A-10E show expression of spliced and unspliced Xv1. FIG. 10A is an mRNA analysis showing expression of unspliced and spliced (Xv1s) forms of Xv1 mRNA in cancer cell lines. FIG. 10B illustrates positions of specific primers used for Xv1s and Xv1u used in the mRNA analysis shown in FIG. 10A. FIG. 10C shows an immunoblotting analysis of Xv1 in cancer cell lines. FIG. 10D shows immunoblotting of Xv1s in BT474 cells transfected with two different Xv1 siRNAs. FIG. 10E shows an immunoblotting analysis of the effect of proteasome and autophagy inhibitors on endogenous Xv1u and Xv1s levels.

FIGS. 11A-11D show the role of inositol-requiring enzyme-1α (IRE1α) in Xv1 splicing. FIG. 11A shows the effect of ER stress inducer and IRE1α inhibitor on splicing of XBP1 and Xv1. FIG. 11B shows the effect of IRE1β RNase inhibitor on Xv1s expression. FIG. 11C shows the effect of IRE1β siRNA on Xv1s expression. FIG. 11D shows that transfection of Xv1s but not Xv1u cDNA rescues cells from death induced by Xv1 siRNA.

FIGS. 12A-12L show subcellular localization and downstream regulation by Xv1. FIG. 12A is a HA, DAPI and merged fluorescence image for HeLa cells transfected with HA-tagged Xv1u or HA-tagged Xv1s. FIG. 12B shows immunoblotting data for subcellular fractionation of endogenous Xv1s. FIG. 12C shows a representative Gene Set Enrichment Analysis for ectopically overexpress Xv1s. FIG. 12D shows expression of XBP1 target genes DNAJC3, DNAJB9, BiP and HERP1 in Xv1s or XBP1s transfected BT474 and MCF7ca cells. FIG. 12E shows a volcano plot of RNA-seq dataset obtained from HeLa cells transfected with Xv1s cDNA and vector control. FIG. 12F shows RT-PCR analysis of TTLL6 and Xv1 expression. FIG. 12G shows RT-PCR analysis of TTLL6, XBP1 and Xv1 expression. FIG. 12H shows RT-PCR analysis of TTLL6 in BT474 transfected with indicated siRNAs. FIG. 12I shows RT-PCR analysis of TTLL6 in MCF7ca transfected with indicated siRNAs. FIG. 12J shows caspase 3/7 activation in BT474 cells. FIG. 12K shows caspase 3/7 activation in MCF7ca cells. FIG. 12L shows rescue of Xv1 knockdown-induced cell death in BT474 cells by transfection of TTLL6 cDNA.

FIGS. 13A-13B show the effect of Xv1 knockdown on polyglutamylation, structure, dynamics of spindle microtubule (MT), and mitotic progression. FIG. 13A shows TTLL6, microtubule (MT), DAPI and merged images of cells incubated with negative control siRNA or TTLL6 siRNA. FIG. 13B shows that knockdown of Xv1 or TTLL6 increases mitotic index in BT474 and HeLa cells, tested two days after siRNA transfection.

FIG. 14 shows PI, α-Tubulin staining and a merged images for cells transfected with negative control siRNA, Xv1 siRNA, XBP1 siRNA and TTLL6 siRNA.

FIG. 15 shows representative immunofluorescence staining for polyE, microtubule (MT), DAPI and merged images for cells transfected with negative control siRNA, Xv1 siRNA, XBP1 siRNA and TTLL6 siRNA.

FIGS. 16A-16C show quantitation of the effects Xv1 knockdown on polyglutamylation and mitotic progression. FIG. 16A shows that Xv1 or TTLL6 knockdown decreases MT polyglutamylation in BT474 cells. FIG. 16B shows the effect of Xv1 and TTLL6 knockdown on polyE to MT ratios. FIG. 16C shows the effect of Xv1 and TTLL6 knockdown on polyE to MT ratios, similar to the data in FIG. 16B.

FIGS. 17A-17B show EB1 and microtubule staining in siRNA transfected cells. FIG. 17A shows EB1, microtubule (MT), DAPI staining and merged images for cells transfected with negative control siRNA, Xv1 siRNA, XBP1 siRNA and TTLL6 siRNA. FIG. 17B show quantitation of EB1 to Microtubule ratios from the fluorescence data shown in FIG. 17A.

FIGS. 18A-18B show acetyl-α-tubulin (Ac-α-Tub) and centromere staining in siRNA transfected cells. FIG. 18A shows Ac-α-Tub, centromere (MT), DAPI staining and merged images for cells transfected with negative control siRNA, Xv1 siRNA, XBP1 siRNA and TTLL6 siRNA. FIG. 18B show quantitation of Ac-α-Tub to Centromere ratios from the fluorescence data shown in FIG. 18A.

FIG. 19 shows microtubule (MT), centromere, DAPI staining and merged images for cells transfected with negative control siRNA, Xv1 siRNA, XBP1 siRNA and TTLL6 siRNA.

FIG. 20 illustrates the IRE1β-Xv1-TTLL6 pathway that operates specifically in cancer cells to promote mitosis.

FIGS. 21A-21C shows expression of XBP1 isoforms in chemoresistant acute myeloid leukemia (AML). FIG. 21A shows that isoform 4 (Xv1) is overexpressed in AML patients who failed in induction therapy. FIG. 21B is a magnification of the section in FIG. 21A as shown. FIG. 21C shows induction of Xv1 expression in U937 cells.

FIGS. 22A-22C show X-box protein expression in cancer cell lines. FIG. 22A is an RT-PCR showing XBP1 and Xv1 mRNA expression in AML cell lines. FIG. 22B is an RT-PCR showing XBP1 and Xv1 mRNA expression in pancreatic adenocarcinoma cell lines. FIG. 22B is an RT-PCR showing XBP1 and Xv1 mRNA expression in the indicated cell lines.

FIGS. 23A-23B show the effect of ER stress on splicing of XBP1 mRNA. FIG. 23A shows mRNA expression of spliced XBP1 and Xv1 after tunicamycin induced ER stress. FIG. 23B shows mRNA expression of XBP1u and XBP1s after tunicamycin induced ER stress.

FIG. 24 shows HA and DAPI staining and merged images showing cellular localization of unspliced and spliced forms of Xv1.

FIGS. 25A-25C show mitochondrial localization of Xv1. FIG. 25A shows the sequence of the N-terminal amino acids 1-36 of Xv1 that bears the mitochondrial targeting domain, fused to a green fluorescent protein (GFP) fusion construct (N36-GFP). FIG. 25B shows GFP, Mitotracker, DAPI fluorescence and merged images in control non-transfected cells (top row) and cells transfected with N36-GFP (bottom row). FIG. 25C shows Mitotracker fluorescence and Xv1s colocalization, DAPI counterstaining and merged images for controls (top row) and tested cells (bottom row).

FIGS. 26A-26C shows regulation of oxidative phosphorylation pathway proteins by X-box proteins. FIG. 26A shows regulation of Cytochrome b-c1 complex subunit 2 (III-UQCRC2) by Xv1 for the indicated cell lines. FIG. 26B shows regulation of the indicated oxidative phosphorylation pathway proteins by Xv1 in HeLa. FIG. 26C shows regulation of the indicated oxidative phosphorylation pathway proteins by Xv1 in U251.

FIGS. 27A-27C shows upregulation of tubulin and p53 in XBP1, Xv1 or TTLL6 knockdown cells. FIG. 27A shows upregulation of tubulin and p53 in HeLa transfected with XBP1, Xv1 or TTLL6 siRNA. FIG. 27B shows upregulation of tubulin and p53 in HeLa transfected with XBP1, Xv1 or TTLL6 siRNA. FIG. 27C shows upregulation of α-tubulin in XBP1, Xv1 or TTLL6 knockdown cells.

FIGS. 28A-28C shows downregulation of tubulin in Xv1 overexpressing cells. FIG. 28A shows α-Tubulin levels in HeLa cells after overexpression of Xv1. FIG. 28B is a second experiment showing α-Tubulin levels in cells overexpressing Xv1. FIG. 28C shows α-Tubulin levels in HepG2 and Huh7 cells after overexpression of Xv1.

FIGS. 29A-29D shows the effects of Xv1 knockdown on TTLL6 expression. FIG. 29A shows downregulation of tubulin in Xv1 overexpressing cells. FIG. 29B shows knockdown of TTLL6 by specific siRNA. FIG. 29C shows that knockdown of TTLL6 increased tubulin and PKM2 expression. FIG. 29D shows activation of caspase 3/7 in U251 cells transfected with TTLL6 siRNA.

### DETAILED DESCRIPTION OF THE INVENTION

As used herein, the term “a” or “an” when used in conjunction with the term “comprising” in the claims and/or the specification may mean “one,” but it is also consistent with the meaning of “one or more,” “at least one,” and “one or more than one.” Some embodiments of the invention may consist of or consist essentially of one or more elements, method steps, and/or methods of the invention. It is contemplated that any method described herein can be implemented with respect to any other method described herein.

As used herein, the term “or” in the claims is used to mean “and/or” unless explicitly indicated to refer to alternatives only or the alternatives are mutually exclusive, although the disclosure supports a definition that refers to only alternatives and “and/or.”

As used herein, “comprise” and its variations, such as “comprises” and “comprising,” will be understood to imply the inclusion of a stated item, element or step or group of items, elements or steps but not the exclusion of any other item, element or step or group of items, elements or steps unless the context requires otherwise. Similarly, “another” or “other” may mean at least a second or more of the same or different claim element or components thereof.

As used herein, the term “contacting” refers to any method suitable for delivering an inhibitor of a cancer biomarker into contact with a cancer cell or tissue or vasculature comprising the same. In vitro or ex vivo this is achieved by exposing the cancer cell or the tissue or vasculature to the inhibitor in a suitable medium. For in vivo applications, any known method of administration is suitable.

In one embodiment of the present invention, there is provided an inhibitor for decreasing cellular levels or activities of X-box binding protein 1 variant (Xv1) in a cancer cell.

In this embodiment, the inhibitor decreases cellular levels or activities of Xv1.

In this embodiment, any inhibitor including, but not limited to a nucleic acid, a peptide and a small molecular weight compound, or a combination of these inhibitors is used. Further in this embodiment, when the inhibitor is a nucleic acid, any nucleic acid-based inhibitor including, but not limited to a small interfering RNA (siRNA), a Morpholino, a micro RNA (miRNA), a Piwi-interacting RNA (piRNA), a heterogeneous nuclear RNA (hnRNA), a small nuclear RNA (snRNA), a guide RNA (gRNA), a single guide RNA (sgRNA), a endoribonuclease-prepared small interfering RNA (esiRNA), a short hairpin RNA (shRNA), a clustered regularly interspaced short palindromic repeats (CRISPR)-based loss-of-function inhibitor, or an antisense oligonucleotide may be used. A combination of these inhibitors may also be used. Also, the sequence of the nucleic acid is selected from the first exon of Xv1 and its flanking region in the genome DNA sequence or mRNA sequence of Xv1. In one aspect, the inhibitor is a nucleic acid, or nucleic acid-based inhibitor that contains nucleotide sequences derived from the first exon of Xv1 and its flanking region (Table 1, SEQ ID NOS: 1-2) in the genomic DNA sequence or cDNA sequence. In another aspect the nucleic acid sequence of the inhibitor mRNA sequence of Xv1 (Table 2, SEQ ID NOS: 3-42 and Table 3, SEQ ID NOS: 43-45).

TABLE 1
Specific sequences for nucleic acid-
based inhibitors to target Xv1

| SEQ ID | | DNA sequence |
|---|---|---|
| NO | Target | (5′ to 3′) |
| 1 | XvTs first exon | CCGGACTGACCGGAT |
| | (uppercase) and | CCGCCACGCTGGGAA |
| | its 3′-term | CCTAGGGCGGCCCAG |
| | flanking | GGCTCTTTTcTGTAC |
| | sequence | TTTTTAACTCTCTCG |
| | (lowercase) in Xv1 | TTAGAGATGACCAGA |
| | cDNA | GCTGGGGATGCGGGC |
| | | ACCTGTCTTCCAGGC |
| | | CCTCTTGCTGTGTGG |
| | | CCGCAGACTGGTGGT |
| | | TCAGCCTCTTAACTC |
| | | GGACATGAGgaaact |
| | | gaaaaacagagtagc |
| | | agctcagactgccag |
| | | agatc-3′ |
| 2 | Xv1 first exon | CCGGACTGACCGGAT |
| | (uppercase) and | CCGCCACGCTGGGAA |
| | its 3′-term | CCTAGGGCGGCCCAG |
| | flanking | GGCTCTTTTCTGTAC |
| | sequence | TTTTTAACTCTCTCG |
| | (lowercase)in | TTAGAGATGACCAGA |
| | genomicDNA | GCTGGGGATGCGGGC |
| | sequence | ACCTGTCTTCCAGGC |
| | | CCTCTTGCTGTGTGG |
| | | CCGCAGACTGGTGGT |
| | | TCAGCCTCTTAACTC |
| | | GGACATGAGgtcgaa |
| | | taatctgttttggtt |
| | | tactgctatttctgg |
| | | agaggcgcggagct |

TABLE 2
siRNA sequences for reducing Xv1 levels

| SEQ ID NO | Target | siRNA sequence |
|---|---|---|
| 3 | Xv1 | GGAUCCGCCACGCUGGGAA |
| 4 | Xv1 | CCGCCACGCUGGGAACCUA |
| 5 | Xv1 | CGCCACGCUGGGAACCUAG |
| 6 | Xv1 | CGGCCCAGGGCUCUUUUCU |
| 7 | Xv1 | GCCCAGGGCUCUUUUCUGU |
| 8 | Xv1 | CCCAGGGCUCUUUUCUGUA |
| 9 | Xv1 | CCAGGGCUCUUUUCUGUAC |
| 10 | Xv1 | AGGGCUCUUUUCUGUACUU |
| 11 | Xv1 | GGGCUCUUUUCUGUACUUU |
| 12 | Xv1 | GGCUCUUUUCUGUACUUUU |
| 13 | Xv1 | GCUCUUUUCUGUACUUUUU |
| 14 | Xv1 | ACUUUUUAACUCUCUCGUU |
| 15 | Xv1 | CUUUUUAACUCUCUCGUUA |
| 16 | Xv1 | ACUCUCUCGUUAGAGAUGA |
| 17 | Xv1 | CUCUCUCGUUAGAGAUGAC |
| 18 | Xv1 | CUCUCGUUAGAGAUGACCA |
| 19 | Xv1 | CUCGUUAGAGAUGACCAGA |
| 20 | Xv1 | CGUUAGAGAUGACCAGAGC |
| 21 | Xv1 | AGAUGACCAGAGCUGGGGA |
| 22 | Xv1 | GGAUGCGGGCACCUGUCUU |
| 23 | Xv1 | UGCGGGCACCUGUCUUCCA |
| 24 | Xv1 | CUGUCUUCCAGGCCCUCUU |
| 25 | Xv1 | CUUGCUGUGUGGCCGCAGA |
| 26 | Xv1 | GCCGCAGACUGGUGGUUCA |
| 27 | Xv1 | CAGACUGGUGGUUCAGCCU |
| 28 | Xv1 | GACUGGUGGUUCAGCCUCU |
| 29 | Xv1 | ACUGGUGGUUCAGCCUCUU |
| 30 | Xv1 | UGGUGGUUCAGCCUCUUAA |
| 31 | Xv1 | GGUGGUUCAGCCUCUUAAC |
| 32 | Xv1 | GUGGUUCAGCCUCUUAACU |
| 33 | Xv1 | GGUUCAGCCUCUUAACUCG |
| 34 | Xv1 | AGCCUCUUAACUCGGACAU |
| 35 | Xv1 | GCCUCUUAACUCGGACAUG |
| 36 | Xv1 | CCUCUUAACUCGGACAUGA |
| 37 | Xv1 | CUCUUAACUCGGACAUGAG |
| 38 | Xv1 | UCGGACAUGAGGAAACUGA |
| 39 | Xv1 | CGGACAUGAGGAAACUGAA |
| 40 | Xv1 | GGACAUGAGGAAACUGAAA |
| 41 | Xv1 | GACAUGAGGAAACUGAAAA |
| 42 | Xv1 | AUGAGGAAACUGAAAAACA |

In another embodiment of the present invention, there is provided a method for decreasing a X-box binding protein 1 variant (Xv1) in a cancer cell comprising contacting the cancer cell with the Xv1 inhibitor described above.

In this embodiment, the cancer cell is from a solid tumor or a disseminated cancer. Examples of such cancers include, but are not limited to, a mammary gland cancer, a hepatocellular cancer, a pancreatic cancer, a glioma, a lung cancer, a colon cancer, a sarcoma, a bladder cancer, a leukemia and a head and neck cancer.

In yet another embodiment of the present invention, there is provided a pharmaceutical composition for treating a cancer comprising an X-box binding protein 1 variant (Xv1) inhibitor; and at least one pharmaceutically acceptable carrier.

In this embodiment, any inhibitor including, but not limited to, a nucleic acid, a peptide and a small molecular weight compound, or a combination of these inhibitors is used. Further in this embodiment, when the inhibitor is a nucleic acid, any nucleic acid-based inhibitor including, but not limited to, a small interfering RNA (siRNA), a Morpholino, a micro RNA (miRNA), a Piwi-interacting RNA (piRNA), a heterogeneous nuclear RNA (hnRNA), a small nuclear RNA (snRNA), a guide RNA (gRNA), a single guide RNA (sgRNA), a endoribonuclease-prepared small interfering RNA (esiRNA), a short hairpin RNA (shRNA), a clustered regularly interspaced short palindromic repeats (CRISPR)-based loss-of-function inhibitor, or an antisense oligonucleotide may be used. A combination of these inhibitors may also be used. Also, the sequence of the nucleic acid is selected from the first exon of Xv1 and its flanking region in the genome DNA sequence (Table 1) or mRNA sequence of Xv1. In one aspect, the inhibitor is a nucleic acid, or nucleic acid-based inhibitor that contains nucleotide sequences derived from the first exon of Xv1 and its flanking region (Table 1, SEQ ID NOS: 1-2) in the genomic DNA sequence or cDNA sequence or mRNA sequence of Xv1 (Table 2, SEQ ID NOS: 3-42 and Table 3, SEQ ID NOS: 43-45).

Also in this embodiment, in one aspect, the pharmaceutically acceptable carrier is a biologically compatible inert solvent including, but not limited to, a water, a buffer, an isotonic saline, an alcohol and a dimethyl sulfoxide. A combination of these solvents may be employed. Alternatively, the pharmaceutically acceptable carrier is a virus, a liposome, an extracellular vesicle, or a polymer suspended in the biologically compatible inert solvent.

In yet another embodiment of the present invention, there is provided a method for treating a cancer in a subject in need thereof comprising administering to the subject a pharmaceutically acceptable amount of the pharmaceutical composition described above.

In this embodiment, the cancer is a solid tumor or a disseminated cancer. Examples of such cancers include, but are not limited to, a mammary gland cancer, a hepatocellular cancer, a pancreatic cancer, a glioma, a lung cancer, a colon cancer, a sarcoma, a bladder cancer, a leukemia and a head and neck cancer.

Further to this embodiment, the method comprises administering to the subject at least one additional anti-cancer drug. In this embodiment, the anti-cancer drug is different from the Xv1 inhibitor and comprises any drug including, but not limited to, a nucleic acid, a peptide and a small molecular weight compound. A combination of these drugs may also be employed.

In yet another embodiment of the present invention, there is provided a kit for targeting an X-box binding protein 1 variant (Xv1) comprising at least one Xv1 inhibitor; at least one pharmaceutically acceptable carrier; a means for detecting the Xv1 protein or mRNA thereof; and instructions for using the kit.

In this embodiment, any inhibitor including, but not limited to a nucleic acid, a peptide and a small molecular weight compound, or a combination of these inhibitors is used. Further in this embodiment, when the inhibitor is a nucleic acid, any nucleic acid-based inhibitor including, but not limited to a small interfering RNA (siRNA), a Morpholino, a micro RNA (miRNA), a Piwi-interacting RNA (piRNA), a heterogeneous nuclear RNA (hnRNA), a small nuclear RNA (snRNA), a guide RNA (gRNA), a single guide RNA (sgRNA), a endoribonuclease-prepared small interfering RNA (esiRNA), a short hairpin RNA (shRNA), a clustered regularly interspaced short palindromic repeats (CRISPR)-based loss-of-function inhibitor, or an antisense oligonucleotide may be used. A combination of these inhibitors may also be used. Also, the sequence of the nucleic acid is selected from the first exon of Xv1 and its flanking region in the genome DNA sequence (Table 1) or mRNA sequence of Xv1. In one aspect, the inhibitor is a nucleic acid, or nucleic acid-based inhibitor that contains derived from the first exon of Xv1 and its flanking region (Table 1, SEQ ID NOS: 1-2) in the genomic DNA sequence or cDNA sequence or mRNA sequence of Xv1 (Table 2, SEQ ID NOS: 3-42 and Table 3, SEQ ID NOS: 43-45).

In this embodiment, in one aspect, the pharmaceutically acceptable carrier is a biologically compatible inert solvent including, but not limited to a water, a buffer, an isotonic saline, an alcohol and a dimethyl sulfoxide. A combination of these solvents may be employed. Alternatively, the pharmaceutically acceptable carrier is a virus, a liposome, or a polymer suspended in the biologically compatible inert solvent.

Also in this embodiment, in one aspect the means for detecting the Xv1 protein is an antibody that enables detection using methods including, but not limited to, immunocytochemistry, ELISA and western blotting. In another aspect, the means for detecting the Xv1 protein is by detecting presence of its corresponding mRNA using methods including, but not limited to in situ hybridization and RT-PCR.

The following examples are given for the purpose of illustrating various embodiments of the invention and are not meant to limit the present invention in any fashion.

### Example 1

### Cell Culture

Human primary dermal fibroblast (HDFa) was purchased from ATCC (PCS-201-012). Normal untransformed fibroblast GM05294 was obtained from Coriell Institute. CWR-R1 and MCF7ca (MCF7 human breast cancer cells transfected with aromatase gene) were obtained from Dr. Yun Qiu (University of Maryland, Baltimore). Huh7 was obtained from Dr. Hongbing Wang (University of Maryland, Baltimore). MDA-MB-231, MDA-MB-453, SKBR3, Hs578T, BT474, T47D, SU8686 and MiaPaCa2 cell lines were obtained from NIH. MV4-11 and MOLM-14 were obtained from Dr. Chengkui Qu (Emery University). Other cell lines were obtained from ATCC.

All cell lines were grown at 37° C. with 5% CO2. HDFa was maintained in Fibroblast Basal Medium obtained from American Type Culture Collection (ATCC-PCS-201-030) supplemented with Fibroblast Growth Kit-Serum-free (ATCC-PCS-201-040. MV4-11 MOLM-14, SU8686 CWR-R1 were maintained in RPM11640 supplemented with 10% fetal bovine serum (FBS). GM05294 was maintained in Minimum Essential Medium (MEM) supplemented with 10% FBS. MCF10A was maintained in Dulbecco's Modified Eagle's medium (DMEM)/F12 supplemented with 5% horse serum, human epidermal growth factor (20 ng/mL), hydrocortisone (500 μg/mL), insulin (10 μg/mL), cholera toxin (100 ng/mL). Other cell lines were maintained in DMEM supplemented with 10% FBS.

### Antibodies

Antibodies to α-tubulin (DM1A, #3873) and Caspase 3 (#9668) were purchased from Cell signaling technology. Antibody to DNAJB9 (13157-1-AP) and HRP-conjugated α-Tubulin antibody (HRP-66031) were purchased from Proteintech. Antibodies to PARP-1 (sc-8007) and acetylated α-tubulin (sc-23950) were purchased from Santa Cruz biotechnology. Anti-β-actin-peroxidase antibody (A3854) and TTLL6 antibody (HPA052397) were purchased from Sigma. Anti-centromere antibodies (15-235) were purchased from Antibodies Incorporated. Anti-polyglutamate chain (polyE) antibody (AG-25B-0030) were purchased from Adipogen. EB1 antibody (ab53358) was purchased from Abcam. HERP1 antibody (TA507019) was purchased from Origene. BiP antibody (610978) was purchased from BD Transduction Laboratories. DNAJC3 antibody (MA5-14820) was purchased from Invitrogen. Anti-Histone H3 (C-terminus) antibody (819412) was purchased from Biolegend. Anti-HA (3F10) antibody (11867423001) was purchased from Roche. Ire1β antibody (MBS9210486) was purchased from MyBioSource. HRP or Alexa Fluor dye conjugated secondary antibodies were all purchased from Invitrogen.

Polyclonal antibodies (UMY162): To produce antibodies that can recognize all XBP1 (unspliced and spliced) and Xv1 (unspliced and spliced) proteins, a common peptide of these proteins (RQRLGMDALVAEEEAEAC, SEQ ID NO: 49 was synthesized by Peptide 2.0 Inc. (Chantilly, Va.). A cysteine was added to the C-terminus for conjugation of the peptide to keyhole limpet hemocyanin (KLH) to improve the immunogenicity. UMY162 was purified from rabbit antiserum using the same peptide that was biotinylated at the C-terminus and bound to streptavidin magnetic beads (Thermo scientific, Waltham, Mass.).

### RNA Interference

Negative Control #1 siRNA was purchased from Ambion. All other siRNAs were synthesized by Sigma, including (Table 3) Xv1 (SEQ ID NO: 44), Xv1 (SEQ ID NO: 45), XBP1 (SEQ ID NO: 46), polyglutamylase Tubulin Tyrosine Ligase Like 6 (TTLL6, SEQ ID NO: 47), TTLL6 (SEQ ID NO: 48) and IRE1β (SEQ ID NO: 49). Lipofectamine RNAiMAX (Invitrogen) was used to transfect siRNAs.

TABLE 3
siRNA sequences for knocking
down Xv1, TTLL6 and IRE1β

| SEQ ID NO | Target | siRNA sequence |
|---|---|---|
| 43 | Xv1 | CCUCUUAACUCGGACAUGAdTdT |
| | (sense) | |
| 44 | Xv1 | CUCUCGUUAGAGAUGACCAdTdT |
| | (sense) | |
| 45 | XBP1 | GACCCCUAAAGUUCUGCUUdTdT |
| | (sense) | |
| 46 | TTLL6 | GAAACGUAUGAGAAGGAAAdTdT |
| | (sense) | |
| 47 | TTLL6 | GCUUUGCGACGACCUCUUAdTdT |
| | (sense) | |
| 48 | IRE1 p | GGGAUUAAUGAAACUGCCAdTdT |
| | (sense) | |

### Plasmid Constructs

Xv1 cDNA with a HA-tag coding sequence fused to the 3′ end of Xv1s ORF was synthesized by Gene Universal Inc. and subcloned into pLVX-EF1α-IRES-puro (Clontech). To express XBP1 and TTLL6, XBP1 and TTLL6 cDNAs were amplified by RT-PCR from U251 cDNA and cloned into pLVX-EF1α-IRES-puro. To make pLVX-XBP1s and pLVX-Xv1s-HA, the 26 base pair (bp) intron sequence was removed by Q5 Site-Directed Mutagenesis Kit (NEB). To make pLVX-Xv1u that cannot be spliced by IRE1, two silent mutations was made by QuikChange II Site-directed mutagenesis kit (Agilent) in IRE1 recognition sites, which breaks the stem-loop structure for IRE1 recognition 38. pLVX-Xv1u-HA were constructed by PCR. For Xv1 RNAi rescue experiments, plasmids harboring Xv1 siRNA (Xv1si)-resistant Xv1, Xv1s, or Xv1u-HA cDNAs, were constructed by site-directed mutagenesis. All constructs and mutations were verified by DNA sequencing. The primers are listed in Table 4.

TABLE 4
Primer sequences

| | SEQ | | |
|---|---|---|---|
| | ID | Pri- | Primer |
| Application | NO | mer | sequence |
| XBP1 RT-PCR | 50 | P1 | GAGGAGAAGGC |
| | | | GCTGAG |
| | 51 | P3 | CACTGGCCTCA |
| | | | CTTCATTC |
| Xv1 RT-PCR | 52 | P2 | GGTGGTTCAGC |
| | | | CTCTTAACTC |
| | 53 | P3 | CACTGGCCTCA |
| | | | CTTCATTC |
| Xv1s RT-PCR | 54 | P2 | GGTGGTTCAGC |
| | | | CTCTTAACTC |
| | 55 | P4 | CTGCACCTGCT |
| | | | GCGT*ACTC |
| Xv1u RT-PCR | 56 | P2 | GGTGGTTCAGC |
| | | | CTCTTAACTC |
| | 57 | P5 | GAGGTGCACGT |
| | | | AGTCTGAG |
| β-actin RT-PCR | 58 | P6 | CACCAACTGGG |
| | | | ACGACAT |
| | 59 | P7 | ACAGCCTGGAT |
| | | | AGCAACG |
| TTLL6 RT-PCR | 60 | P8 | GCGAAGCCCTT |
| | | | CAGTTCTC |
| | 61 | P9 | GCTCCTCTCAC |
| | | | ATCCTTTTTGG |
| Site-directed | 62 | P10 | GGTGCAGGCCC |
| Xv1 | | | AGTTGTC |
| mutagenesis to | 63 | P11 | TGCTGCGGACT |
| create | | | CAGCAGAC |
| Site-directed | 64 | P12 | CGGGTCTGCA*GAA* |
| Xv1u that will | | | TCCGCAGCAC |
| not be | | | |
| spliced | | | |
| mutagenesis to | 65 | P13 | GTGCTGCGGAT*TCT* |
| create | | | GCAGACCCG |
| Site-directed | 66 | P14 | CCTCTTAACTCT*GA |
| siRNA | | | T*ATGAGGAAACTG |
| (SEQ ID NO: 3) | | | |
| resistant Xv1 | | | |
| mutagenesis | 67 | P15 | CAGTTTCCTCATA* |
| to create | | | TCA*GAGTTAAGAGG |
| PCRXv1u-HA | 68 | P16 | CTTTGCTAGCCACCA |
| | | | TGCGGGCACCTGTCTT |
| | | | C |
| | 69 | P17 | GTTTGCGGCCGCTTA |
| | | | AGCGTAATCTGGAAC |
| | | | ATCGTATGGGTAGTT |
| | | | CATTAATGGCTTCCA |
| | | | GC |
| PCR Non- | 70 | P16 | CTTTGCTAGCCACCA |
| tagged Xv1 | | | TGCGGGCACCTGTCT |
| | | | TC |
| | 71 | P18 | GAAAGGATCCgTTAGA |
| | | | CACTAATCAGCTGGG |
| PCR TTLL6 | 72 | P19 | CTTCACTAGTGCAGCC |
| | | | AATGGGAGCGTTACT |
| | | | CCTTCATC |
| | 73 | P20 | GACTTGCGGCCGCTTA |
| | | | GCTCCTCTCACATCC |
| | | | TTTTTGG |
| PCRXBP1 | 74 | P21 | GTTCACTAGTGCGTAG |
| | | | TCTGGAGCTATG |
| | 75 | P22 | CTTTGGATCCAGACAG |
| | | | GCTTCTCTGCTATC |

*nucleotide before asterix is mutated

### Reverse Transcription Polymerase Chain Reaction (RT-PCR) and Quantitative PCR

Total RNA was prepared using Trizol reagent (Invitrogen) according to the manufacturer's protocol. Reverse transcription was performed using PROTOSCRIPT First Strand cDNA Synthesis Kit (New England BioLabs) using 2 μg total RNA/reaction. Primers used in RT-PCR are listed in Table 4.

To assess Xv1 expression level in normal breast tissues and in tumors at different breast cancer stages, qPCR was performed in Breast Cancer cDNA Array I (Origene, BCRT101) using a CFX96 Touch Real-Time PCR Detection System (Bio-rad). The cDNA array contains 48 samples covering 7-normal, 10-Stage I, 20-stage II, 11-Stage III. The qPCR reactions were set up for detection of Xv1 or β-actin in a total volume of 30 μL using IQ SYBR Green Supermix (Bio-rad). Primer sets used for Xv1 and β-actin have similar amplification efficiencies. To calculate the relative Xv1 level for each sample, the threshold cycle (Ct) value for Xv1 was normalized to the value for β-actin (ΔCt=Ct(Xv1)-Ct(β-actin)). The relative Xv1 levels were calculated as 2ΔCt for each sample. To plot the fold changes of different tumor stages over normal tissue, the mean of the relative Xv1 levels in normal tissues was arbitrary set as 1.

### RNA-Seq

HeLa cells were transfected with vector or pLVX-Xv1s by Lipofectamine 2000 (Invitrogen). Two days after transfection, total RNA was prepared from these cells using Trizol reagent (Invitrogen) according to the manufacturer's protocol. The RNA samples were sent to Novogene Corporation Inc. for quality control and RNA-seq services. Skewer was used to trim adapters from paired end reads, and HISAT2 was used to align sequences to Homo sapiens NCBI reference genome assembly version GRCh38 (39-41). The align reads were counted and assigned gene features using featureCounts as a part of the Subread package 41. Analysis of counts were conducted using the R programming language and Bioconductor libraries including edgeR, limma, and Gene Set Variation Analysis (GSVA, 20,42,43). After counts were transformed and normalized, preprocessing for modeling was conducted using the voom-limma procedure prior to differential expression analysis. GSVA enrichment was performed with MSigDB defined gene sets 20,21.

### Database Analysis

The relative expression levels (isoform percentage, IsoPct) of each XBP1 isoform in the transcriptomic datasets Genotype-Tissue Expression (GTEx) and the Cancer Genome Atlas (TCGA) were analyzed using the TCGA TARGET GTEx study (13). GTEx has expression data from normal tissues whereas TCGA contains the data from tumors and a limited number of normal tissues near the tumors. The overall survival analysis was performed using GEPIA2 (14). For this analysis, Xv1 levels were normalized to the expression levels of XBP1 gene (all 7 variants), which is similar as isoform percentage.

### Live Cell Analysis

Real-time apoptosis was monitored using an IncuCyte@ S3 Live-Cell Analysis System (Sartorius). Cells were seeded to 96 well plates. The next day, the culture medium was replaced with fresh one containing IncuCyte@ Caspase 3/7 Green reagent (1:2000 final), which generates green fluorescence once cleaved by activated caspase 3/7. The cells were then transfected with different siRNAs with Lipofectamine RNAiMAX. Green fluorescence was monitored by time-lapse imaging and normalized to real-time cell confluence. For rescue experiments, cells were first transfected with plasmids using X-tremeGENE HP DNA Transfection Reagent (Roche). Sixteen hours after transfection, the cells were seeded to 96-well plates and then followed the aforementioned protocol for siRNA transfection and caspase 3/7-activity monitoring.

To monitor mitotic changes in siRNA-transfected cells, BT 474 cells were seeded in 96-well plates and transfected with siRNA the next day. Two days after siRNA transfection, IncuCyte@ Caspase 3/7 Green reagent (1:2000) and INCUCYTE NucLight Rapid Red Reagent (1:800) that stains DNA were added to the cells by medium changing before the starting of time-lapse imaging using short intervals (e.g. 15 min).

### Nuclear-Cytoplasmic Fractionation

BT474 cells were treated with DMSO or bortezomib (BTZ, 2 μM) for 2 hours. The nuclear and cytoplasmic fractions were prepared from these cells using NE-PER Nuclear and Cytoplasmic Extraction Reagents (Thermo) following the manufacturer's protocol.

### Microtubule Isolation

Microtubule isolation was performed as previously reported (44), with some modifications. BT474 cells (˜1×106) transfected with different siRNAs for 2 days were lysed in large volume (2 mL) of PEM buffer (100 mM 1,4-piperazine-bis-ethane sulfonic acid (PIPES), pH 6.9, 5 mM MgCl2, 1 mM ethylene glycol tetraacetic acid (EGTA)) with 0.5% Triton X-100 and 10 μM paclitaxel for 10 min at 37° C. After centrifugation at 20,000×g for 10 min, the supernatant containing free tubulin was removed. The pellet containing microtubules was briefly washed with PEM buffer and then lysed directly in sampling buffer for immunoblotting.

### Immunofluorescence

For better labeling efficiencies, different fixation conditions were applied to different primary antibodies. In all cases, Alexa Fluor 488 or 594-conjugated secondary antibodies were used to label the primary antibodies at room temperature for 1 h. Nuclei were labeled with DAPI. Fluorescent microscopy was performed using a Zeiss Axiovert 200M fluorescent microscope.

To analyze the localization of Xv1s and Xv1u, HeLa cells growing on coverslips were transfected with pLVX-Xv1s-HA or pLVX-Xv1u-HA using Lipofectamine 2000. 24 h after transfection, the cells were washed with PBS and fixed in 4% paraformaldehyde (PFA) for 30 min at room temperature. The cells were blocked in 3% BSA and then labeled with rat monoclonal anti-HA antibody (3F10, 1:1000).

To analyze the effects of knockdown Xv1 or TTLL6 on polyglutamylation of MT, BT474 cells transfected with different siRNAs for 2 days were fixed with an optimized method to better preserve MT structures (45). Briefly, the cells were prefixed in protein crosslinking reagent dithiobis (succinimidylpropionate) (DSP) for 10 min at 37° C. Then the cells were permeabilized with 0.5% Triton X-100 in MT-stabilizing buffer (MTSB, 1 mM EGTA, 4% PEG8000, 100 mM PIPES, pH6.9) for 10 min at 37° C. followed by fixation with 4% PFA in MTSB for 15 min at 37° C. After blocking in 5% goat serum in PBS for 1 h, the cells were labeled with rabbit polyE antibody (1:1000) and α-tubulin antibody (DM1A, 1:800) for overnight at 4° C. To label the localization of TTLL6, BT474 cells were fixed as for polyE staining. Rabbit polyclonal TTLL6 antibody (1:50) and mouse monoclonal α-tubulin antibody (DM1A, 1:800) were used to labeled TTLL6 and the microtubules (MT) for overnight at 4° C.

For EB1 staining, BT474 cells transfected with different siRNAs for 2 days were also prefixed in DSP for 10 min at 37° C. and then fixed in cold methanol for 10 min at −20° C. Rat monoclonal EB1 antibody (1:500) was co-stained with α-tubulin antibody (DM1A, 1:800) for overnight at 4° C.

To co-stain acetylated α-tubulin and kinetochore, BT474 cells transfected with different siRNAs for 2 days were fixed in 4% paraformaldehyde (PFA) in culture media for 20 min at 37° C. The cells were labeled with mouse monoclonal acetylated α-tubulin antibody (1:500) and anti-centromere antibodies (ACAs, 1:100) derived from human CREST patient serum) for overnight at 4° C.

To analyze cold stabilities of MTs in different knockdown cells, BT474 cells transfected with different siRNAs for 2 days were incubated in precooled PBS for 10 min on ice. Then the cells were fixed at room temperature for 10 min with 4% PFA in 100 mM PIPES pH6.8, 10 mM EGTA, 1 mM MgCl2, 0.2% Triton X-100 46. ACAs and α-tubulin antibody (DM1A) were then used to label the kinetochores and MTs.

To analysis mitotic index, BT474 and HeLa cells transfected with different siRNAs for 2 days were fixed with cold methanol for 20 min at −20° C. MT was label with α-tubulin antibody (DM1A, 1:800) and the nucleus was labeled with propidium iodide (PI).

### Tumorigenicity Assays

At 40 hours after transfection, no viability changes were observed with propidium iodide staining in BT474 cells transfected with different siRNAs with Lipofectamine RNAiMAX. RNAi efficiencies were confirmed by RT-PCR. Cells were then used for soft agar colony formation assay and xenograft tumor assay. Soft agar colony formation assay was performed in 6-well dishes. The bottom layer contained 1.5 mL 0.5% agar in culture medium. 5,000 BT474 cells transfected with different siRNAs were diluted in 1 mL of 0.3% agar in culture medium and laid on top of the solidified bottom layer. Three replicated wells were used for each condition. The plates were placed to 37° C. humidified cell culture incubator after the top layer solidified. After 4 weeks, the colonies in 5 random fields were counted under microscope for each well.

Xenograft experiment was performed by Translational Shared Service at University of Maryland School of Medicine. BT474 cells transfected with negative control #1, Xv1 or XBP1 siRNA were washed once with 1×PBS. Six-week old female Nu/nu mice (Envigo, Frederick Md.) were injected subcutaneously with 3×10⁶ cells on both flanks (n=5 mice/siRNA=10 tumors/siRNA). Tumor initiation and growth was followed over time. Tumor volume was measured with electronic calipers. Animals were euthanized at day 32 when tumors were excised, weighed. Tumor tissue sectioning for pathological examination was performed by Pathology Biorepository Shared Service, University of Maryland School of Medicine. Xenograft tumor tissues were fixed in 4% paraformaldehyde and processed for paraffin embedding and sectioning. Hematoxylin & eosin (H&E) was performed on 5-μm-thick paraffin sections.

### Statistical Analysis

Statistical significance was assessed by paired or unpaired two-tailed Student's t-test using GraphPad Prism 7.0. For all analyses, P>0.05 was considered not significant (n.s.), whereas P≤0.05 (*), P≤0.01 (**) and P≤0.001 (***) are considered statistically significant.

### Example 2

### X-Box Binding Protein Variants—a Cancer-Specific, Alternative Spliced Form of XBP1 (Xv1)

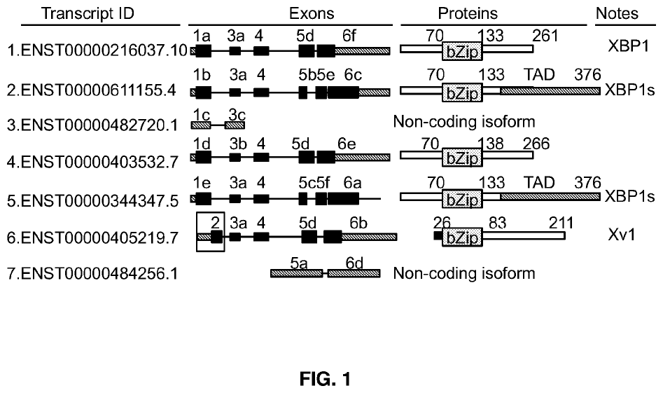

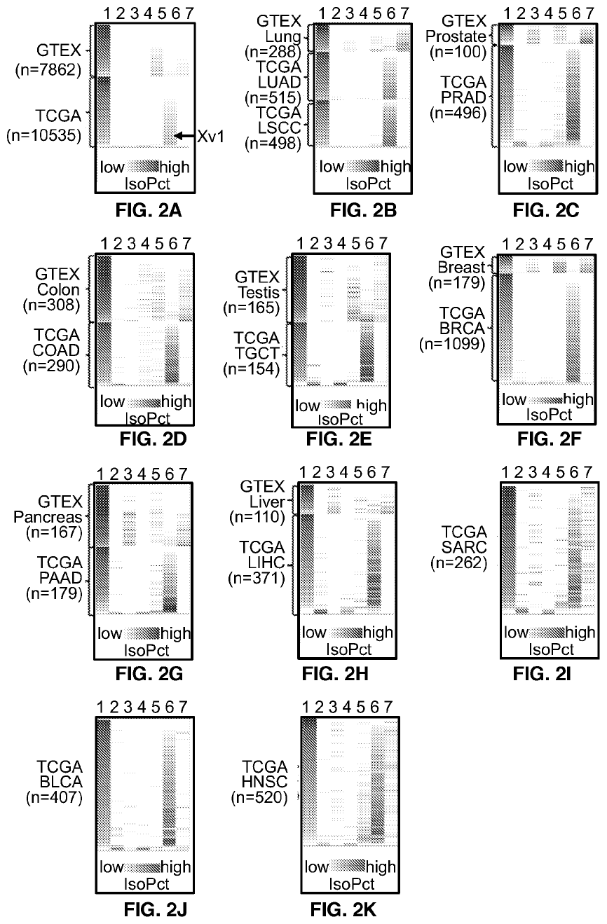

XBP1 is a key mediator of the unfolded protein response (UPR) that signals to protect normal cells against endoplasmic reticulum (ER) stress. In malignant cells, XBP1 signaling is reprogramed to promote tumor growth, chemoresistance, and metastasis as well as evading anti-tumor immunity (3,6,11,12). XBP1 has seven different transcript variants as revealed by mining the transcriptomic datasets in UCSC Genome Browser (13). FIG. 1 shows a schematic of XBP1 variant transcripts and proteins, where the box for transcript 6 denoting the cryptic exon 2 in Xv1. Xv1 lacks exon 1 of the XBP1 gene but contains a cryptic first exon (exon 2) that is unique to Xv1 and conserved only in humans and great apes. Of the various variants, Xv1 (ENST00000405219.7) is a previously underappreciated variant that is highly expressed in many cancers. FIGS. 2A-2K show relative expression level (isoform percentage, IsoPct) of each XBP1 isoform in the transcriptomic datasets in Genotype-Tissue Expression (GTEX, normal tissue expression data) and The Cancer Genome Atlas (TCGA) for Lung adenocarcinoma (LUAD), Lung squamous cell cancer (LSCC), Prostate adenocarcinoma (PRAD), Colon adenocarcinoma (COAD), Testicular Germ Cell Tumor (TGCT), breast cancer (BRCA), pancreatic cancer (PAAD), Liver hepatocellular carcinoma (LIHC), Sarcoma (SARC), Bladder Urothelial Carcinoma (BLCA) and Head and Neck squamous cell carcinoma (HNSC).

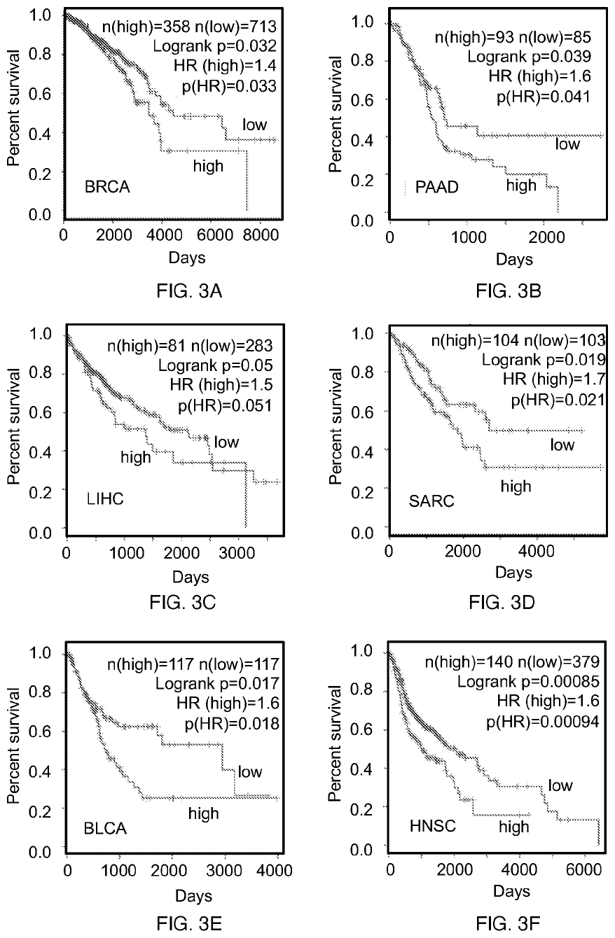

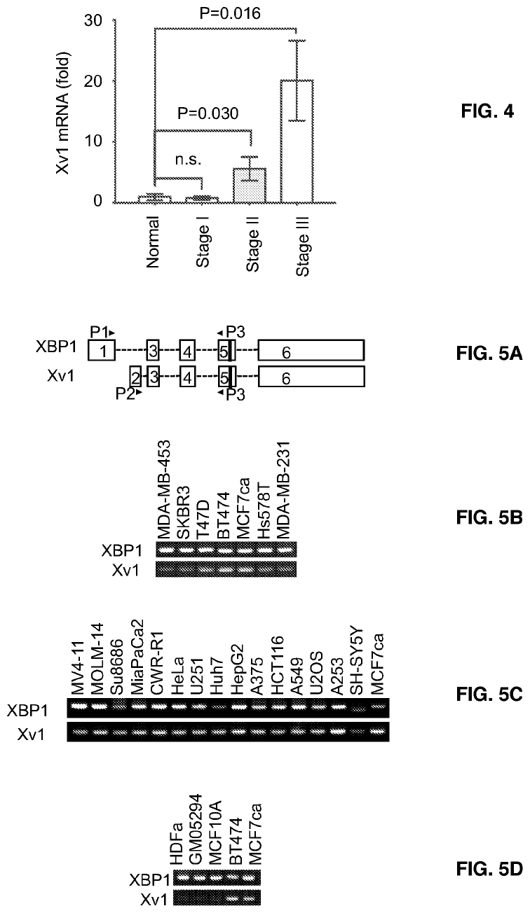

Survival analysis using Gene Expression Profiling Interactive Analysis (GEPIA2) tool (14) revealed that high abundance of Xv1 transcript (FIGS. 2A-2K) is associated with poor survival of patients with breast cancer (BRCA), pancreatic cancer (PAAD), liver cancer (LIHC, Liver hepatocellular carcinoma), sarcoma (SARC), bladder cancer (BLCA, Bladder Urothelial Carcinoma), and head and neck cancer (HNSC, Head and Neck squamous cell carcinoma) (FIGS. 3A-3F). Xv1 levels normalized to total XBP1 gene expression levels were used in the overall survival analysis. Further, high abundance of Xv1 mRNA expression in breast cancer tissues by quantitative RT-PCR revealed a cancer stage-dependent progressive increase in Xv1 expression (FIG. 4). FIGS. 5A-5D shows that Xv1 mRNA is widely detected in various types of cancer cell lines (FIGS. 5B and 5C) but not in non-cancerous cells (FIG. 5D), including human primary dermal fibroblast (HDFa), fibroblasts derived from normal skin (GM05294), and MCF10A that was derived from pre-cancerous mammary epithelial cell. Arrows in FIG. 5A show the positions of the specific RT-PCR primers for XBP1, and Xv1 (undetected in non-cancerous cells).

### Example 3

### Effect of Xv1 Knockdown on Cell Survival

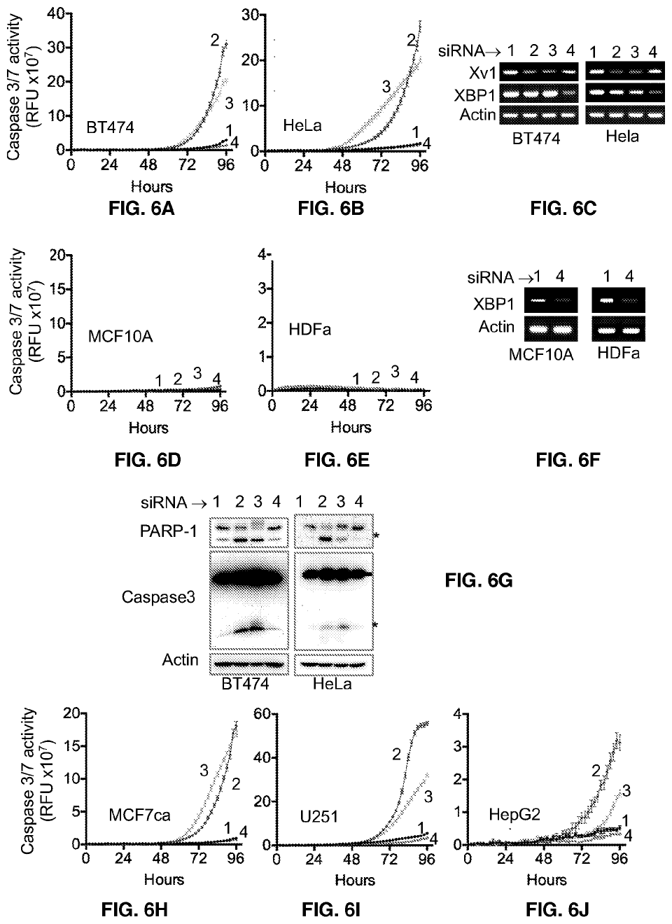

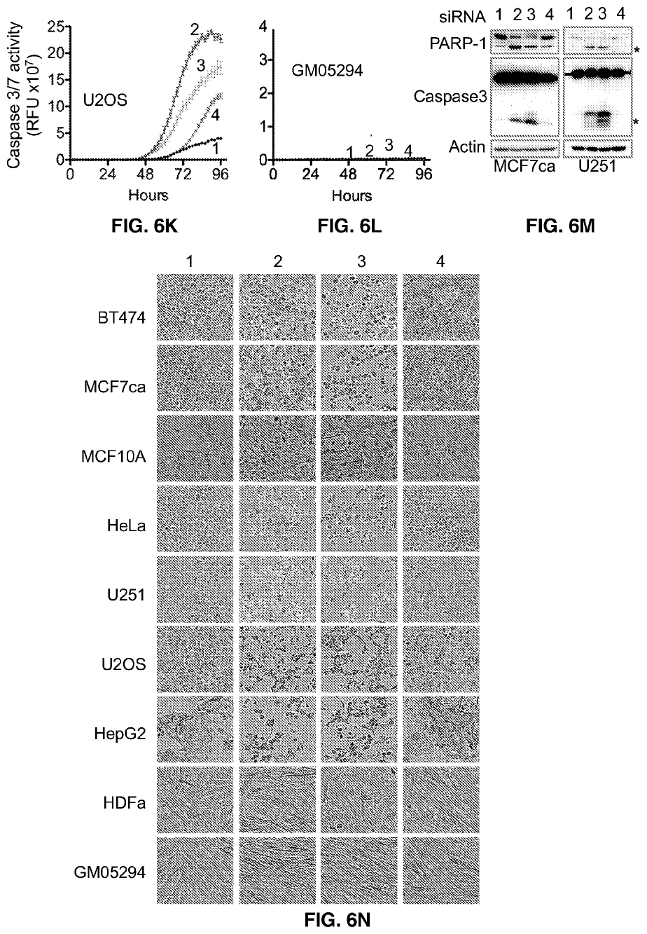

To determine the role of Xv1 in cell survival, siRNA directed knockdown of Xv1 was performed. FIGS. 6A-6C and 6N show that compared to negative control siRNA (1), Xv1 knockdown (2, SEQ ID NO: 3 and 3, SEQ ID NO: 4) induced apoptosis in breast cancer (BT474) and cervical cancer (HeLa) cells as revealed by caspase 3/7 activation in live cells. In contrast, XBP1 knockdown (4, SEQ ID NO: 5) had minimal effects on cancer cell viability. Moreover, Xv1 siRNA transfection did not induce death of non-cancerous cells, including HDFa and MCF10A, that do not express Xv1 (FIGS. 6D-6F and 6N). Immunoblotting for cleaved caspase 3 and PARP-1 confirmed that knockdown of Xv1 but not XBP1 induced apoptosis in cancer cells (FIG. 6G, asterisks indicate the cleaved forms of caspase 3 and PARP-1). Similar observations were made in other cancer cell lines and normal human skin fibroblasts (FIGS. 6H-6M and 6N).

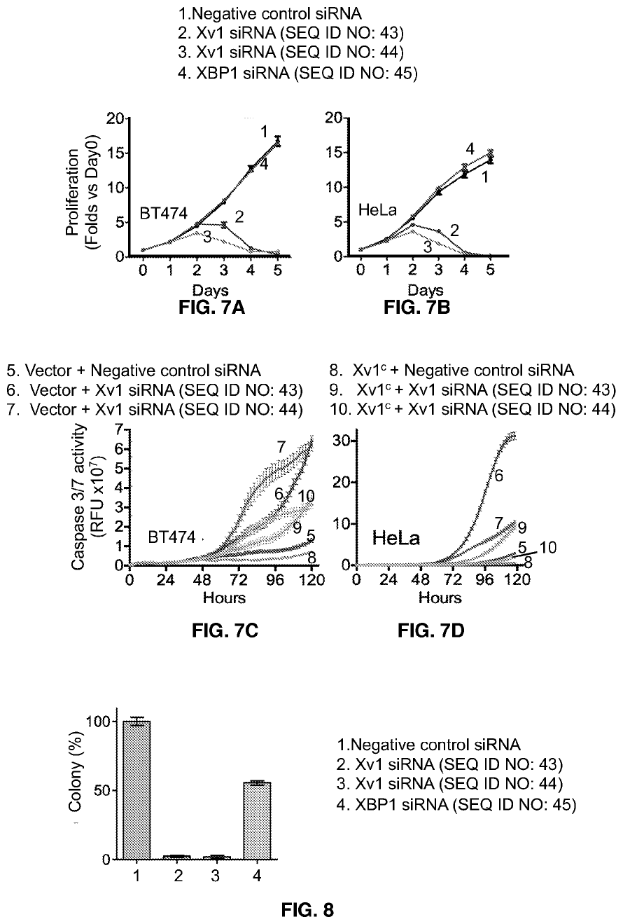

Consistent with induced apoptosis, decreases in cell survival were observed in BT474 and HeLa cells depleted of Xv1 (FIGS. 7A and 7B). Importantly, transfection of siRNA resistant Xv1 cDNA (Xv1^{c}) rescued cells from death induced by the Xv1 siRNAs in these cells (FIGS. 7C and 7D). Taken together, the above results suggest a specific requirement of Xv1 for survival of cancer cells, but not normal cells.

To determine the effects of Xv1 on tumorigenicity, soft agar colony formation assay was performed using BT474 cells 40-hours post siRNA transfection when no differences in viabilities could be detected by propidium iodide staining. FIG. 8 shows that Xv1 knockdown markedly suppressed anchorage-independent growth of BT474 cells, whereas knockdown of XBP1 had a smaller effect.

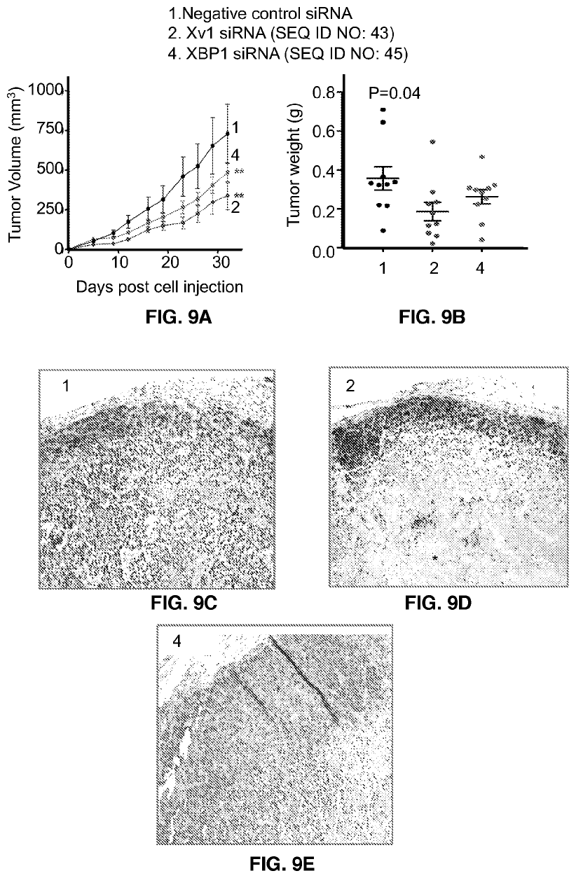

To test the effect of Xv1 knockdown on tumor growth in vivo, a mouse xenograft model was used. siRNA-transfected BT474 breast cancer cells were injected subcutaneously into athymic-nude-foxn1nu mice. Time-dependent tumor growth in volume and tumor weight at day 32 were measured and compared with control animals (n=10 tumors per condition). Representative tumors were processed for H&E staining. The data showed that Xv1 knockdown inhibited the growth of BT474 breast cancer in a mouse xenograft model (FIGS. 9A-9E). Pathologically, the Xv1 knockdown xenograft tumors had substantially larger necrotic centers than the control and XBP1 knockdown tumors (FIGS. 9C-9E). These data suggest that Xv1 is also required for tumor growth in vivo and may have a role in promoting cell survival under hypoxia. Although knockdown of XBP1 did not affect the viability of BT474 cells in vitro (FIG. 6A), it did inhibit growth of BT474 breast cancer in xenograft (FIGS. 9A-9B), which is consistent with a previous report (3).

### Example 4

### Xv1 Splice Variants in Cancer

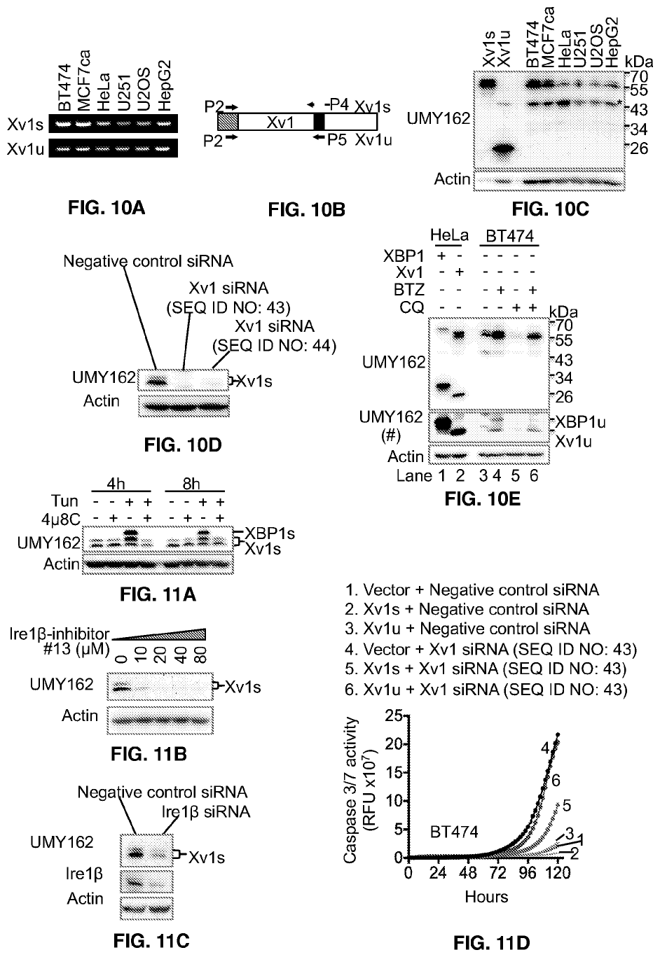

A unique mechanism of XBP1 regulation is the unconventional splicing of XBP1 mRNA to remove a 26-nucleotide intron by IRE1α RNase under ER stress (1,2). Spliced XBP1 mRNA encodes an active transcription factor XBP1s. Since Xv1 mRNA contains the identical 26-nucleotide intron, it was hypothesized that this transcript may also undergo unconventional splicing. Indeed, RT-PCR detected both unspliced (Xv1u) and spliced (Xv1s) forms of Xv1 mRNA, to various extents, in all six cancer cell lines tested (FIG. 10A). FIG. 10B shows the primers for Xv1s (P2+P4) or Xv1u (P2+P5) used for the RT-PCR analysis. Xv1s protein was also detected in these cell lines by immunoblotting with UMY162 (FIG. 10C), a rabbit polyclonal antibody raised against a common peptide in XBP1 (unspliced and spliced) and Xv1 (unspliced and spliced) proteins. The identity of Xv1s was validated by knockdown of Xv1 (FIG. 10D). Importantly, in the same samples, XBP1s was not detected, indicating that cancer cells normally express much higher levels of Xv1s protein than that of XBP1s. Both Xv1u and XBP1u proteins were also not detected by UMY162, which could be due to their instabilities as previously reported for XBP1u and XBP1s proteins (15). Indeed, treatment of BT474 cells with proteasome inhibitor bortezomib (BTZ) but not autophagy inhibitor chloroquine (CQ) increased the levels of Xv1u, Xv1s, and XBP1u proteins but not XBP1s protein (FIG. 10E). These results indicate that Xv1s but not XBP1s is highly expressed in cancer cells, which supports a critical role of Xv1 in cancer cell survival.

### Example 5

### Effect of ER Stress on Xv1 Expression

To determine the role of IRE1α in Xv1 splicing, BT474 cells were treated with tunicamycin to induce ER stress. The treatment increased primarily the XBP1s protein, indicating that XBP1 splicing is inducible by ER stress, whereas Xv1 is constitutively spliced (FIG. 11A). Treatment with an IRE1α inhibitor, 4μ8C, diminished ER stress induced XBP1s but had little effect on basal Xv1s levels (FIG. 11A). IRE1α has a paralog, IRE1β, which shows weak RNase activity towards XBP1 (16-18). Interestingly, inhibition of IRE1β RNase by a reported small molecule inhibitor #13 (19) and knockdown of IRE1βboth decreased the basal levels of Xv1s (FIGS. 11B and 11C). These data suggest that Xv1 is preferentially spliced by IRE1β. Transfection of Xv1s but not Xv1u cDNA (Xv1 siRNA-resistant) rescued BT474 cells from death induced by Xv1 siRNA (FIG. 11D), suggesting that Xv1s but not Xv1u is essential for cancer cell survival.

### Example 6

### Cellular Localization

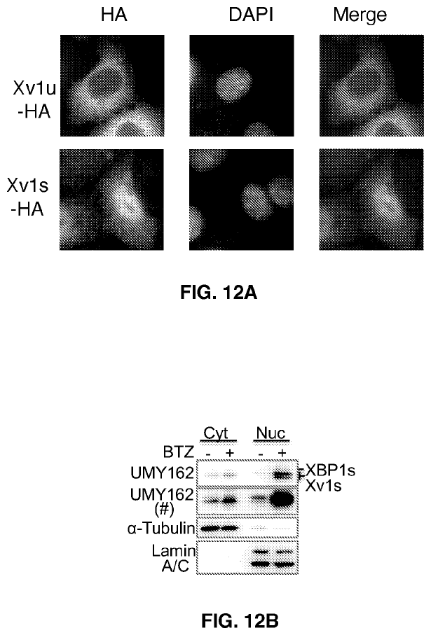

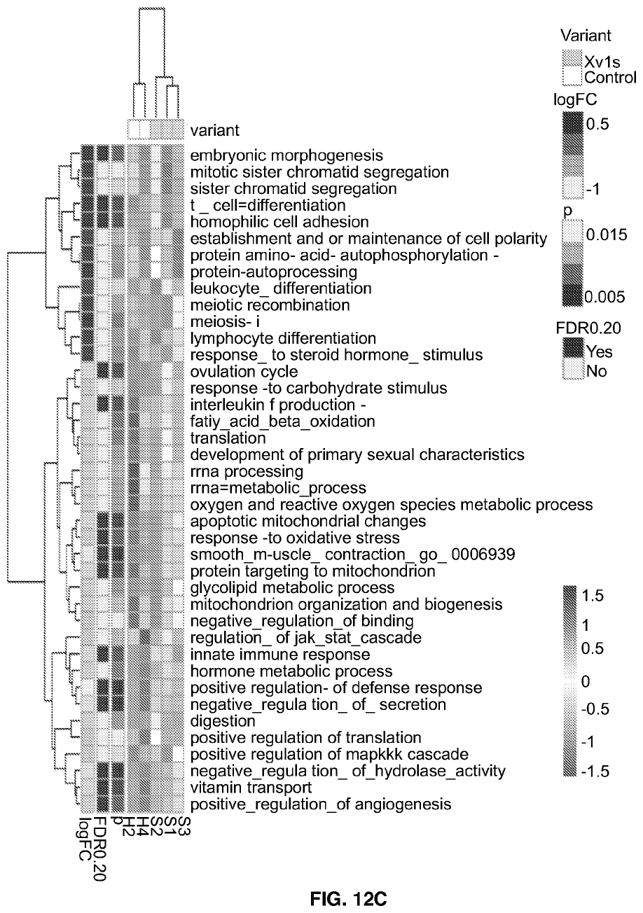

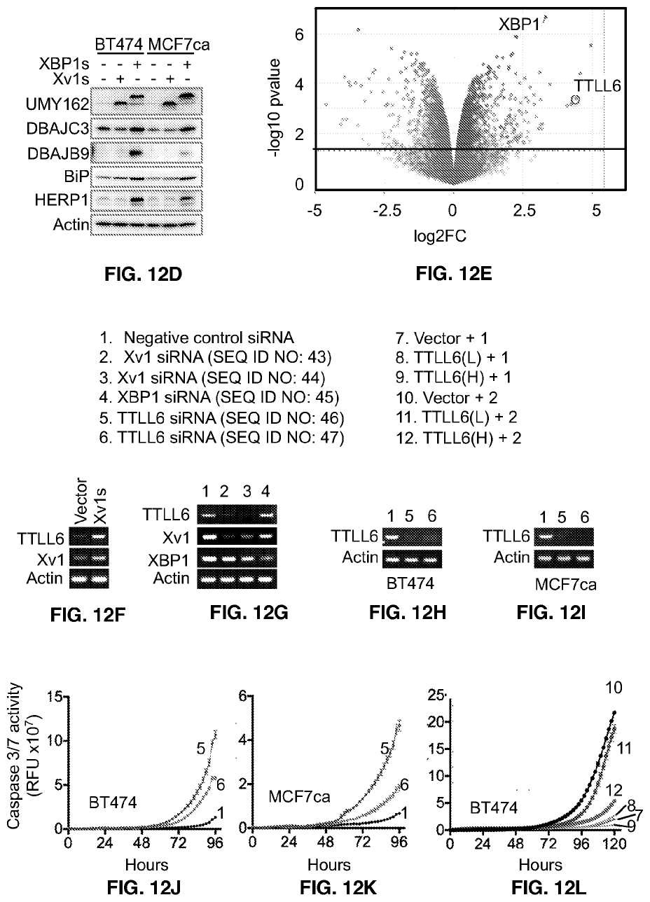

Immunofluorescence revealed that Xv1s was localized in both the cytoplasm and the nucleus, whereas Xv1u was mainly detected in the cytoplasm when ectopically expressed in HeLa cells (FIG. 12A). Subcellular fractionation showed similar subcellular localization of endogenous Xv1s (FIG. 12B). Thus, Xv1s may also act as a transcription factor like XBP1s. To support this interpretation, the transcriptome of HeLa cells that ectopically overexpress Xv1s was analyzed.

Gene set enrichment analysis (20,21) using Gene Set Enrichment Analysis (GSEA) software and Molecular Signature Database (MSigDB). showed that UPR was not among the biological processes affected by Xv1s overexpression (FIG. 12C). Consistently, Xv1s overexpression did not induce the expression of XBP1 target genes such as DNAJC3, DNAJB9, BiP, and HERP1, whereas XBP1s overexpression did (FIG. 12D). Interestingly, the polyglutamylase Tubulin Tyrosine Ligase Like 6 (TTLL6) was identified as one of the top-upregulated genes by Xv1s in the RNA-seq dataset (FIG. 12E). RT-PCR confirmed that overexpression of Xv1s increased TTLL6 expression in BT474 (FIG. 12F). Consistently, knockdown of Xv1 but not XBP1 markedly decreased the levels of TTLL6 mRNA (FIG. 12G). Like Xv1, TTLL6 knockdown also caused death of BT474 and MCF7ca cells (FIGS. 12H-12K). Importantly, Xv1 knockdown-induced BT474 cell death could be rescued by transfection of TTLL6 cDNA in a dose-dependent manner as evidenced by decreased caspase 3/7 activity (FIG. 12L), suggesting that TTLL6 is a key mediator of Xv1 function in cancer cell survival.

### Example 7

### Effect of Xv1 Knockdown on Microtubule Polyglutamylation

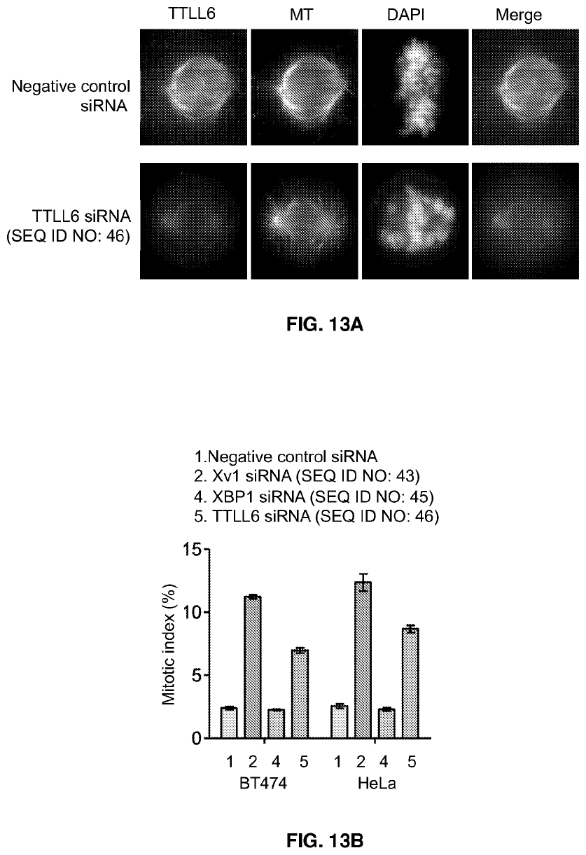

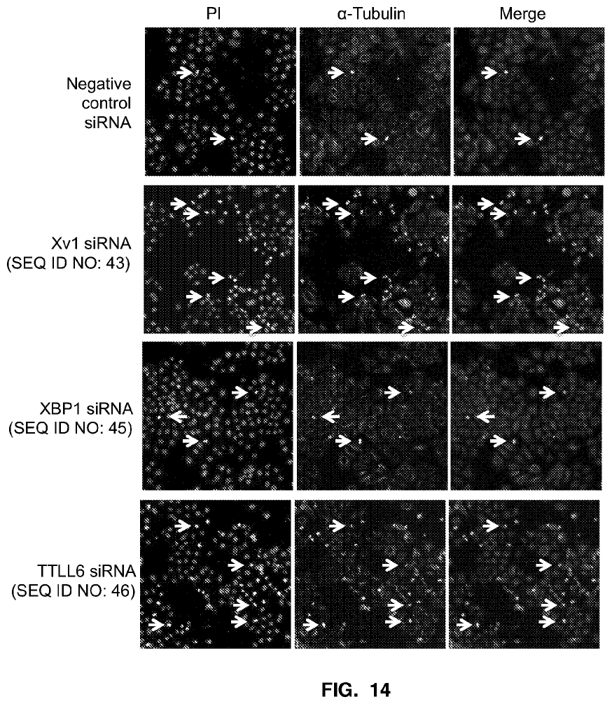

TTLL6 is a polyglutamylase (22,23). Immunofluorescence showed that it was enriched in interpolar spindle microtubules of BT474 cells (FIG. 13A). Knockdown of TTLL6 caused structural defects of the mitotic spindle (FIGS. 13A and 13B), suggesting that it may play a role in mitosis. Therefore, the mitotic index was assessed in BT474 and HeLa cells 48 hours after TTLL6 siRNA transfection when minimal apoptosis has been activated (FIG. 12M). Knockdown of Xv1 and XBP1 were also performed for comparison. The results showed that TTLL6 and Xv1 knockdown increases mitotic index by about three to five folds compared with control knockdown whereas XBP1 knockdown cells had no effect (FIGS. 13B and 14), suggesting that Xv1 regulates mitotic progression through upregulation of TTLL6.

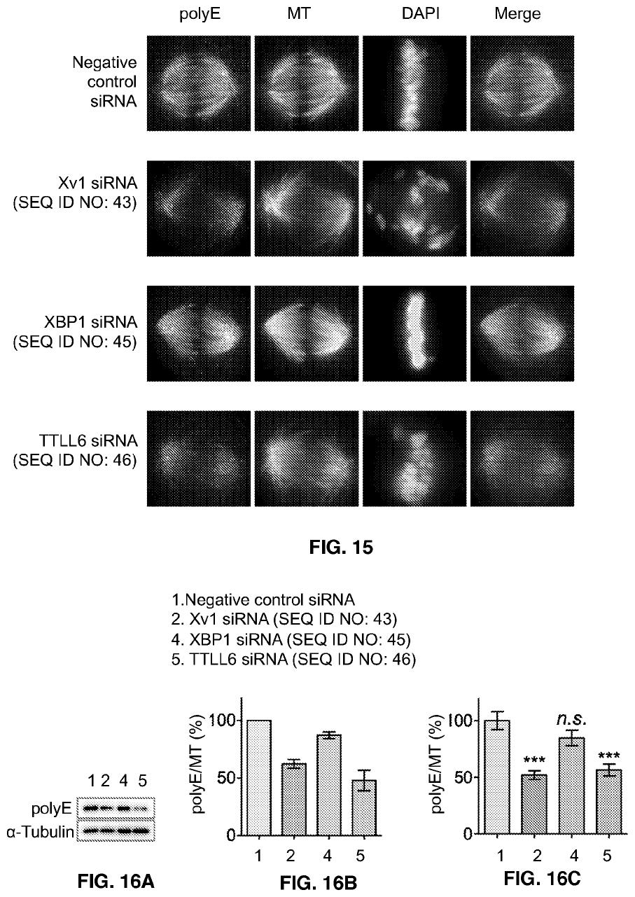

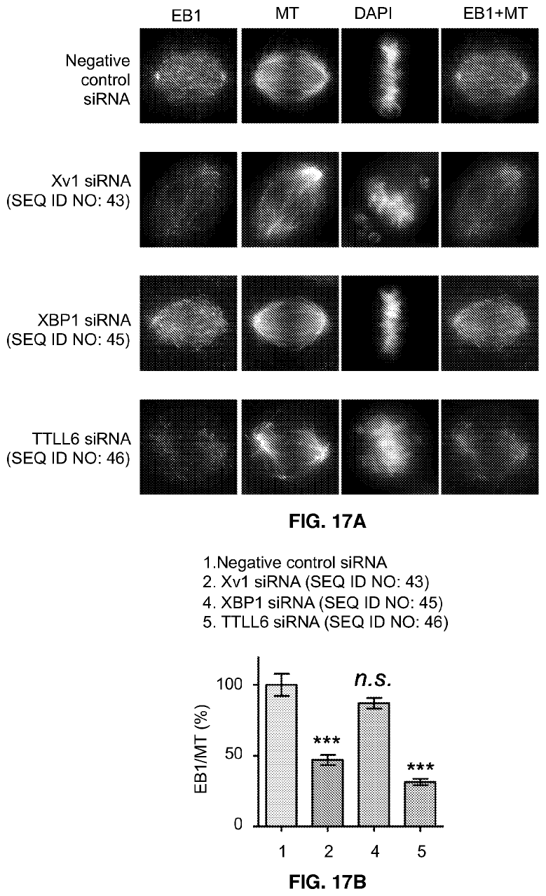

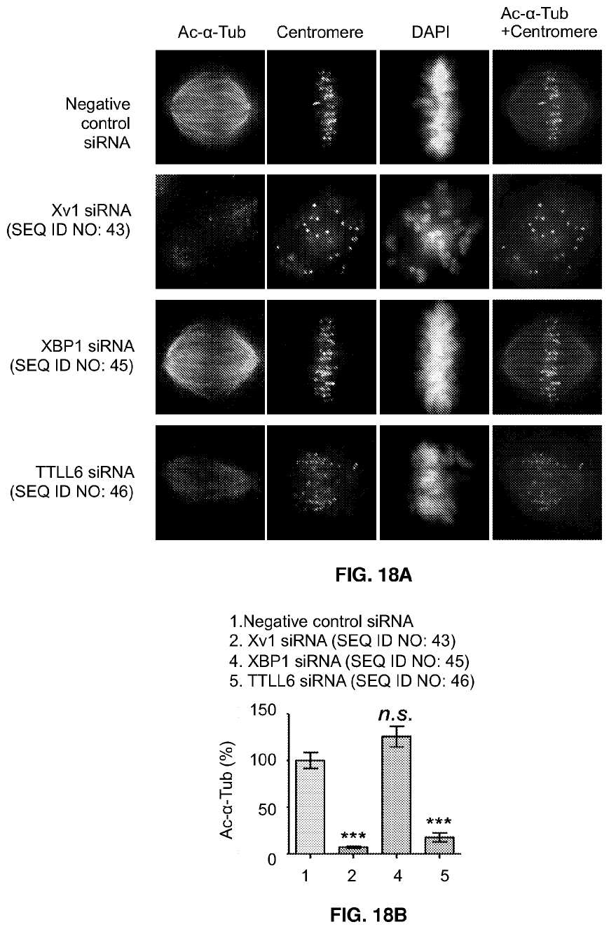

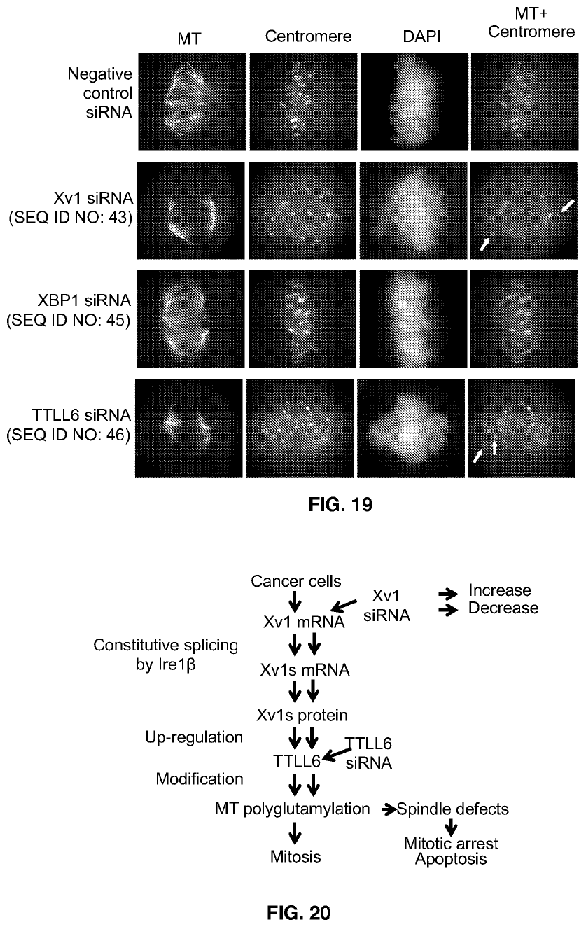

Immunoblotting revealed that microtubule polyglutamylation was decreased in cells with knockdown of either Xv1 or TTLL6 but not XBP1 (FIGS. 15 and 16A-16C). A significant reduction of spindle polyglutamylation was also observed in cells with knockdown of either Xv1 or TTLL6 but not XBP1. These results suggest that TTLL6 catalyzes the polyglutamylation of spindle microtubules. Knockdown of either Xv1 or TTLL6 but not XBP1 also caused other defects of the mitotic spindle, such as decreases in end-binding protein 1 (EB1) binding (which binds to GTP-tubulin and marks the growing plus-ends of microtubules) and tubulin acetylation (an indicator of microtubule stability) throughout the spindle (FIGS. 17A and 17B and 18A-18B), indicating that TTLL6 is required for proper microtubule dynamics and stability. It was also determined that kinetochore-microtubule attachments were destabilized in the absence of either Xv1 or TTLL6 but not XBP1 (FIG. 19). The decrease in polyglutamylation is likely the primary cause for the spindle defects observed. Previous studies have reported that polyglutamylation activates microtubule severing enzymes, such as spastin and katanin that create nanoscale damages throughout the microtubule by active extraction of tubulin heterodimers (24-28). These damage sites are repaired spontaneously by GTP-tubulin incorporation, leading to increases in microtubule number and mass (27,29). The nanoscale damages may also be used as sites for tubulin acetyltransferase (aTAT1) to enter the lumen of microtubules to acetylate tubulin (30-33). Therefore, the Xv1-TTLL6 pathway plays a key role in maintaining spindle structure, dynamics, acetylation, and kinetochore attachment by enhancing spindle polyglutamylation.

The present invention identifies a novel IRE1β-Xv1-TTLL6 pathway which operates specifically in cancer cells to promote mitosis (FIG. 19) and has a critical role and regulation of spindle polyglutamylation in mitotic progression in XV1-positve cancer cells. Mitotic arrest-induced mitotic catastrophe (34) and cGAS activation (35) are likely the causes for death of cancer cells when loss of function of the IRE1β-XVI-TTLL6 pathway. Loss of cell cycle control is a hallmark of cancer and has been an attractive drug target for cancer therapy for decades (36,37). However, cell cycle-targeted drugs in general have a low therapeutic index due to severe toxicity to normal cells (36,37). Therefore, strategies that target cancer cell-specific cycle features are highly desirable (36,37). This Xv1-mediated cancer-specific mitotic regulation provide new opportunities to develop cancer-specific anti-mitosis drugs which overcome the low therapeutic index of the current drugs. In addition, the elevated expression of Xv1 serves as a pharmacogenetic biomarker for drugs that target this novel pathway.

### Example 8

### Xv1 and Acute Myeloid Leukemia (AML) Chemoresistance.

The expression of Xv1 is markedly increased in acute myeloid leukemia (AML) patients who failed in induction therapy (FIGS. 20A and 20B). FIG. 20C shows that Xv1 expression is induced in drug-resistant cultured AML cells (U937). Ctrl: control-parental U937 cells. Ara-C:Cytarabine resistant U937 cells. This data implicates a role for Xv1 expression in conferring AML drug-resistance.

### Example 9

Xv1 mRNA Undergoes Unconventional Splicing Under Endoplasmic Reticulum (ER) Stress

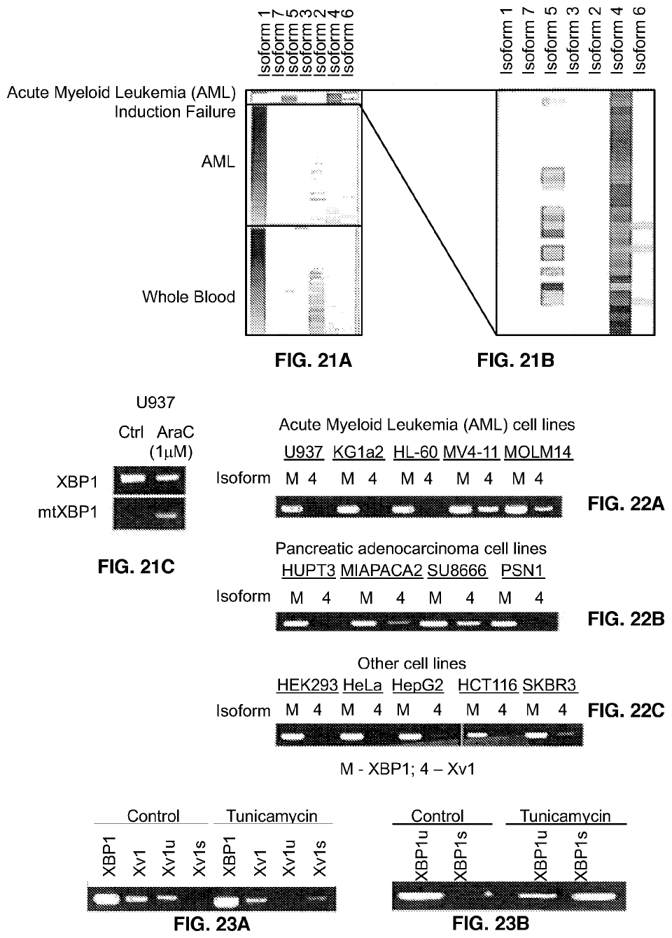

Using RT-PCR, Xv1 mRNA is expressed in various cancer cell lines (FIGS. 21A-21C). XBPI mRNA is known to undergo unconventional splicing under ER stress by Ire1 Xv1 also undergoes splicing in cells under ER stress as revealed by RT-PCR (FIGS. 22A-22B). Cells were treated with tunicamycin to induce ER stress and then total RNAs were isolated for RT-PCR.

### Subcellular Localization of Xv1 Protein

The unspliced form of Xv1 (Xv1u) is localized primarily in the cytoplasm, whereas the spliced Xv1 (Xv1s) is localized in both the cytoplasm and nucleus (FIG. 23).

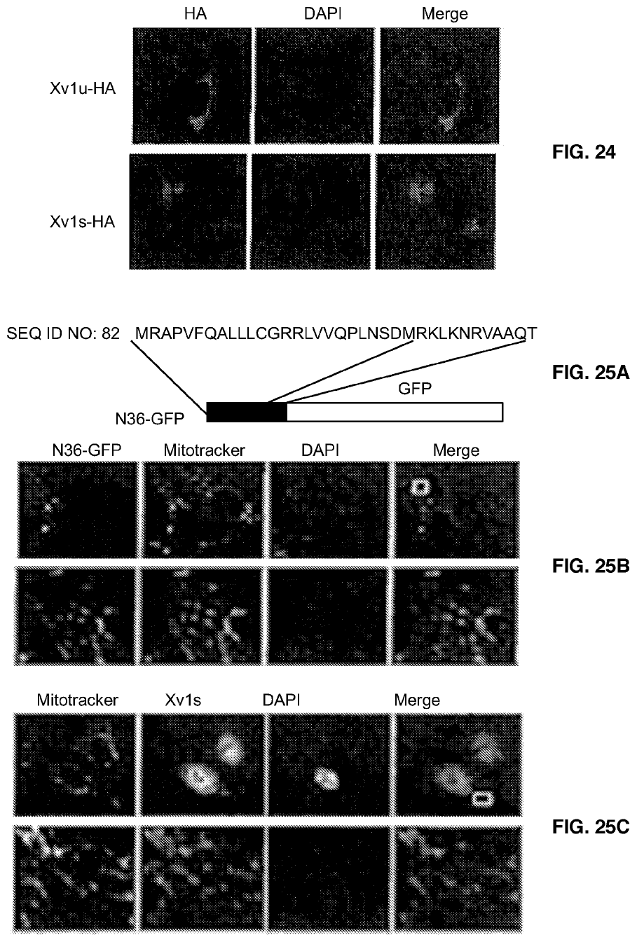

Further, after removal of cytosol by permeabilizing cells with digitonin, a fraction of Xv1 is localized in the mitochondria. Consistently, the N-terminal 25 amino acids of Xv1 (unique to Xv1) is an active mitochondria targeting signal (MTS) (FIGS. 24A and 24B). Xv1s is localized in mitochondria in digitonin-permeabilized HeLa cells (FIG. 24C).

Xv1 Expression Maintains OXPHOS while Suppresses Glycolysis as Revealed by Immunoblotting for Related Proteins

FIGS. 25A-25C shows the down regulation of OXPHOS pathway proteins and upregulation of glycolysis regulators in Xv1 knockdown cells.

### Knockdown of Xv1 or TTLL6 Expression Up Regulates Tubulin

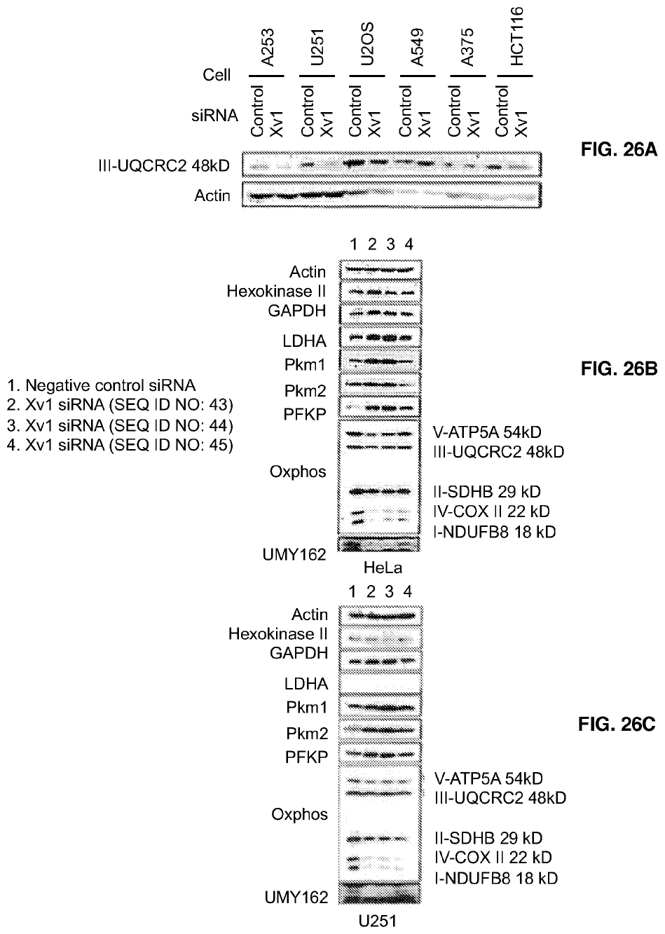

FIGS. 26A-26C shows upregulation of tubulin and p53 in Xv1 or TTLL6 knockdown cells.

### Overexpression of Xv1 Downregulates Tubulin

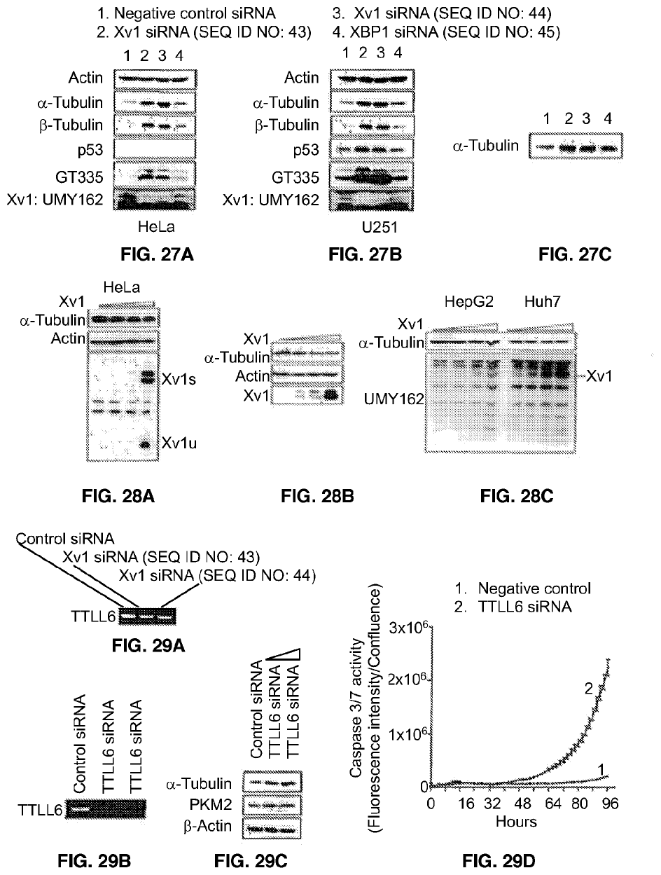

FIGS. 27A-27C shows downregulation of tubulin in Xv1 overexpressing cells.

### TTLL6 is a Xv1 Target Gene and is Essential for Cell Survival

FIG. 28A shows knockdown of Xv1 downregulates TTLL6. FIGS. 28B and 28C show that knockdown of TILL6 increased tubulin and PKM2 expression as seen in Xv1 knockdown cells (see FIGS. 25A-25C and 26A-26C). FIG. 28D. Knockdown of TILL6 causes activation of caspase 3/7 in U251 cells, which is similar to Xv1 knockdown (FIG. 9B, C).

Xv1 mRNA and Protein Sequences The mRNA and protein sequences of Xv1 are shown in Table 5.

TABLE 5
mRNA and Protein Sequences of Xv1

| | SEQ ID | |
|---|---|---|
| Description | NO | Sequence |
| Unique | 76 | CCGGACTGACCGGATCCGCC |
| sequence in Xv1 | | ACGCTGGGAACCTAGGGCGG |
| cDNA | | CCCAGGGCTCTTTTcTGTAC |
| | | TTTTTAACTCTCTCGTTAGA |
| | | GATGACCAGAGCTGGGGATG |
| | | CGGGCACCTGTCTTCCAGGC |
| | | CCTCTTGCTGTGTGGCCGCA |
| | | GACTGGTGGTTCAGCCTCTT |
| | | AACTCGGACAT |
| Full length Xv1 | 77 | CCGGACTGACCGGATCCGCC |
| cDNA | | ACGCTGGGAACCTAGGGCGG |
| | | CCCAGGGCTCTTTTCTGTAC |
| | | TTTTTAACTCTCTCGTTAGA |
| | | GATGACCAGAGCTGGGGATG |
| | | CGGGCACCTGTCTTCCAGGC |
| | | CCTCTTGCTGTGTGGCCGCA |
| | | GACTGGTGGTTCAGCCTCTT |
| | | AACTCGGACATGAGGAAACT |
| | | GAAAAACAGAGTAGCAGCTC |
| | | AGACTGCCAGAGATCGAAAG |
| | | AAGGCTCGAATGAGTGAGCT |
| | | GGAACAGCAAGTGGTAGATT |
| | | TAGAAGAAGAGAACCAAAAA |
| | | CTTTTGCTAGAAAATCAGCT |
| | | TTTACGAGAGAAAACTCATG |
| | | GCCTTGTAGTTGAGAACCAG |
| | | GAGTTAAGACAGCGCTTGGG |
| | | GATGGATGCCCTGGTTGCTG |
| | | AAGAGGAGGCGGAAGCCAAG |
| | | GGGAATGAAGTGAGGCCAGT |
| | | GGCCGGGTCTGCTGAGTCCG |
| | | CAGCACTCAGACTACGTGCA |
| | | CCTCTGCAGCAGGTGCAGGC |
| | | CCAGTTGTCACCCCTCCAGA |
| | | ACATCTCCCCATGGATTCTG |
| | | GCGGTATTGACTCTTCAGAT |
| | | TCAGAGTCTGATATCCTGTT |
| | | GGGCATTCTGGACAACTTGG |
| | | ACCCAGTCATGTTCTTCAAA |
| | | TGCCCTTCCCCAGAGCCTGC |
| | | CAGCCTGGAGGAGCTCCCAG |
| | | AGGTCTACCCAGAAGGACCC |
| | | AGTTCCTTACCAGCCTCCCT |
| | | TTCTCTGTCAGTGGGGACGT |
| | | CATCAGCCAAGCTGGAAGCC |
| | | ATTAATGAACTAATTCGTTT |
| | | TGACCACATATATACCAAGC |
| | | CCCTAGTCTTAGAGATACCC |
| | | TCTGAGACAGAGAGCCAAGC |
| | | TAATGTGGTAGTGAAAATCG |
| | | AGGAAGCACCTCTCAGCCCC |
| | | TCAGAGAATGATCACCCTGA |
| | | ATTCATTGTCTCAGTGAAGG |
| | | AAGAACCTGTAGAAGATGAC |
| | | CTCGTTCCGGAGCTGGGTAT |
| | | CTCAAATCTGCTTTCATCCA |
| | | GCCACTGCCCAAAGCCATCT |
| | | TCCTGCCTACTGGATGCTTA |
| | | CAGTGACTGTGGATACGGGG |
| | | GTTCCCTTTCCCCATTCAGT |
| | | GACATGTCCTCTCTGCTTGG |
| | | TGTAAACCATTCTTGGGAGG |
| | | ACACTTTTGCCAATGAACTC |
| | | TTTCCCCAGCTGATTAGTGT |
| | | CTAAGGAATGATCCAATACT |
| | | GTTGCCCTTTTCCTTGACTA |
| | | TTACACTGCCTGGAGGATAG |
| | | CAGAGAAGCCTGTCTGTACT |
| | | TCATTCAAAAAGCCAAAATA |
| | | GAGAGTATACAGTCCTAGAG |
| | | AATTCCTCTATTTGTTCAGA |
| | | TCTCATAGATGACCCCCAGG |
| | | TATTGTCTTTTGACATCCAG |
| | | CAGTCCAAGGTATTGAGACA |
| | | TATTACTGGAAGTAAGAAAT |
| | | ATTACTATAATTGAGAACTA |
| | | CAGCTTTTAAGATTGTACTT |
| | | TTATcTTAAAAGGGTGGTAG |
| | | TTTTCCCTAAAATACTTATT |
| | | ATGTAAGGGTCATTAGACAA |
| | | ATGTCTTGAAGTAGACATGG |
| | | AATTTATGAATGGTTCTTTA |
| | | TCATTTCTCTTCCCCCTTTT |
| | | TGGCATCCTGGCTTGCCTCC |
| | | AGTTTTAGGTCCTTTAGTTT |
| | | GCTTCTGTAAGCAACGGGAA |
| | | CACCTGCTGAGGGGGCTCTT |
| | | TCCCTCATGTATACTTCAAG |
| | | TAAGATCAAGAATCTTTTGT |
| | | GAAATTATAGAAATTTACTA |
| | | TGTAAATGCTTGATGGAATT |
| | | TTTTCCTGCTAGTGTAGCTT |
| | | CTGAAAGGTGCTTTCTCCAT |
| | | TTATTTAAAA |
| Xv1 mRNA | 78 | CCGGACTGACCGGATCCGCC |
| sequence | | ACGCTGGGAACCTAGGGCGG |
| | | CCCAGGGCTCTTTTCTGTAC |
| | | TTTTTAACTCTCTCGTTAGA |
| | | GATGACCAGAGCTGGGGATG |
| | | CGGGCACCTGTCTTCCAGGC |
| | | CCTCTTGCTGTGTGGCCGCA |
| | | GACTGGTGGTTCAGCCTCTT |
| | | AACTCGGACATGAGGAAACT |
| | | GAAAAACAGAGTAGCAGCTC |
| Unspliced form | 79 | MRAPVFQALLLCGRRLVVQP |
| of human Xv1 | | LNSDMRKLKNRVAAQTARDR |
| protein | | KKARMSELEQQVVDLEEENQ |
| | | KLLLENQLLREKTHGLVVEN |
| | | QELRQRLGMDALVAEEEAEA |
| | | KGNEVRPVAGSAESAALRLR |
| | | APLQQVQAQLSPLQNISPWI |
| | | LAVLTLQIQSLISCWAFWTT |
| | | WTQSCSSNALPQSLPAWRSS |
| | | QRSTQKDPVPYQPPFLCQWG |
| | | RHQPSWKPLMN |
| Spliced form of | 80 | MRAPVFQALLLCGRRLVVQP |
| human Xv1 | | LNSDMRKLKNRVAAQTARDR |
| protein | | KKARMSELEQQVVDLEEENQ |
| | | KLLLENQLLREKTHGLVVEN |
| | | QELRQRLGMDALVAEEEAEA |
| | | KGNEVRPVAGSAESAAGAGP |
| | | VVTPPEHLPMDSGGIDSSDS |
| | | ESDILLGILDNLDPVMFFKC |
| | | PSPEPASLEELPEVYPEGPS |
| | | SLPASLSLSVGTSSAKLEAI |
| | | NELIRFDHIYTKPLVLEIPS |
| | | ETESQANVVVKIEEAPLSPS |
| | | ENDHPEFIVSVKEEPVEDDL |
| | | VPELGISNLLSSSHCPKPSS |
| | | CLLDAYSDCGYGGSLSPFSD |
| | | MSSLLGVNHSWEDTFANELF |
| | | PQLISV |
| N-terminal amino | 81 | MRAPVFQALLLCGRRLVVQP |
| acids 1-25 of Xv1 | | LNSDM |
| protein | | |
| N-terminal amino | 82 | MRAPVFQALLLCGRRLVVQP |
| acids 1-36 of Xv1 | | LNSDMRKLKNRVAAQT |
| protein | | |
| N-terminal amino | 83 | MVVVAAAPNPADGTPKVLLL |
| acids 1-75 of | | SGQPASAAGAPAGQALPLMV |
| XBP1 protein | | PAQRGASPEAASGGLPQARK |
| | | RQRLTHLSPEEKALR |

The following references are cited herein:
1. Yoshida H. et al., Cell, 107:881-891, 2001.
2. Lee, K. et al., Genes Dev, 16:452-466, 2002.
3. Chen, X. et al., Nature, 508:103-107, 2014.
4. Sheng, X. et al., Nat Commun., 10:323, 2019.
5. Song, M. et al., Nature, 562:423-428, 2018.
6. Cubillos-Ruiz, J. R. et al., Cell, 161:1527-1538, 2015.
7. Demircioglu, D. et al., Cell, 178:1465-1477 e1417, 2019.
8. Reyes, A. & Huber, W., Nucleic Acids Res., 46:582-592, 2018.
9. Kahles, A. et al., Cancer Cell, 34:211-224 e216, 2018.
10. Lau, E. et al., Cell Rep., 29:3751-3765 e3755, 2019.
11. Wang, M. & Kaufman, R. J., Nat Rev Cancer, 14:581-597, 2014.
12. Obacz, J. et al., Sci Signal 10(470):eaal2323, 2017.
13. Vivian, J. et al., Nat Biotechnol., 35:314-316, 2017.
14. Tang, Z. et al., Nucleic Acids Res., 47:W556-W560, 2019.
15. Calfon, M. et al., Nature, 415:92-96, 2002.
16. Grey, M. J. et al., J Cell Biol., 219(2):e201904048, 2020.
17. Martino, M. B. et al., Mucosal Immunol., 6:639-654, 2013.
18. Imagawa, Y. et al., FEBS Lett., 582:656-660, 2008.
19. Feldman, H. C. et al., ACS Chem Biol., 14:2595-2605, 2019.
20. Hanzelmann, S. et al., BMC Bioinformatics, 14:7, 2013.
21. Subramanian, A. et al., Proc Natl Acad Sci USA, 102:15545-15550, 2005.
22. Zempel, H. et al., EMBO J., 32:2920-2937, 2013.
23. Janke, C. & Magiera, M. M., Nat Rev Mol Cell Biol, 21:307-326, 2020.
24. McNally, F. J. et al., J Cell Sci, 109 (3):561-567 1996.
25. Hartman, J. J. et al., Cell, 93:277-287, 1998.
26. McNally, F. J. & Roll-Mecak, A., J Cell Biol 217:4057-4069, 2018.
27. Vemu, A. et al., Science, 361, 2018.
28. Lacroix, B. et al., J Cell Biol, 189:945-954, 2010.
29. Kuo, Y. W. et al., Proc Natl Acad Sci USA, 116:5533-5541, 2019.
30. Maruta, H., Greer, K. & Rosenbaum, J. L., J Cell Biol, 103:571-579, 1986.
31. Bulinski, J. C. et al., J Cell Biol, 106:1213-1220, 1988.
32. Akella, J. S. et al., Nature, 467:218-222, 2010.
33. Shida, T. et al., Proc Natl Acad Sci USA, 107:21517-21522, 2010.
34. Vitale, I. et al., Nat Rev Mol Cell Biol, 12:385-392, 2011.
35. Zierhut, C. et al., Cell 178:302-315 e323, 2019.
36. Dominguez-Brauer, C. et al., Mol Cell 60:524-536, 2015.
37. Otto, T. & Sicinski, P., Nat Rev Cancer, 17:93-115, 2017.
38. Hong, S. Y. & Hagen, T., Br J Haematol, 161:898-901, 2013.
39. Jiang, H. et al., BMC Bioinformatics, 15:182, 2014.
40. Kim, D. et al., Nat Methods, 12:357-360, 2015.
41. Liao, Y. et al., Bioinformatics 30:923-930, 2014.
42. Robinson, M. D. et al., Bioinformatics 26:139-140, 2010.
43. Ritchie, M. E. et al., Nucleic Acids Res, 43:e47, 2015.
44. De Conto, F. et al., PLoS One, 7:e41207, 2012.
45. Bell, P. B., Jr. & Safiejko-Mroczka, B., Scanning Microsc, 9:843-857, 1995.
46. Lampson, M. A. & Kapoor, T. M., Nat Cell Biol, 7:93-98, 2005.

What is claimed:

1. An inhibitor for decreasing cellular levels or activities of X-box binding protein 1 variant (Xv1) in a cancer cell.

2. The inhibitor of claim 1, wherein the inhibitor is a nucleic acid, a peptide, or a small molecule compound, or a combination thereof.

3. The inhibitor of claim 2, wherein the inhibitor is a nucleic acid designed from a nucleotide sequence of SEQ ID NO: 1 or SEQ ID NO: 2.

4. The inhibitor of claim 2, wherein the inhibitor is a small interfering RNA (siRNA), a Morpholino, a micro RNA (miRNA), a Piwi-interacting RNA (piRNA), a heterogeneous nuclear RNA (hnRNA), a small nuclear RNA (snRNA), a guide RNA (gRNA), a single guide RNA (sgRNA), a endoribonuclease-prepared small interfering RNA (esiRNA), a short hairpin RNA (shRNA), a clustered regularly interspaced short palindromic repeats (CRISPR)-based loss-of-function inhibitor, or an antisense oligonucleotide, or a combination thereof.

5. The inhibitor of claim 4, wherein the inhibitor is an siRNA comprising at least one nucleotide sequence selected from the group consisting of SEQ ID NOS: 3-45.

6. A method for decreasing an X-box binding protein 1 variant (Xv1) in a cancer cell, comprising contacting the cancer cell with the inhibitor of claim 1.

7. The method of claim 5, wherein the cancer cell is from a solid tumor or a disseminated cancer.

8. The method of claim 7, wherein the cancer cell is from a mammary gland cancer, a hepatocellular cancer, a pancreatic cancer, a colon cancer, a glioma, a lung cancer, a sarcoma, a bladder cancer, a leukemia, or a head and neck cancer.

9. A pharmaceutical composition for treating a cancer, comprising:
an X-box binding protein 1 variant (Xv1) inhibitor; and
at least one pharmaceutically acceptable carrier.

10. The pharmaceutical composition of claim 9, wherein the inhibitor is a nucleic acid, a protein, a peptide, or a small molecular weight compound, or a combination thereof.

11. The pharmaceutical composition of claim 10, wherein the inhibitor is a nucleic acid designed from a nucleotide sequence of SEQ ID NO: 1 or SEQ ID NO: 2.

12. The pharmaceutical composition of claim 10, wherein the nucleic acid is a small interfering RNA (siRNA), a Morpholino, a micro RNA (miRNA), a Piwi-interacting RNA (piRNA), a heterogeneous nuclear RNA (hnRNA), a small nuclear RNA (snRNA), a guide RNA (gRNA), a single guide RNA (sgRNA), a endoribonuclease-prepared small interfering RNA (esiRNA), a short hairpin RNA (shRNA), a clustered regularly interspaced short palindromic repeats (CRISPR)-based loss-of-function inhibitor, or an antisense oligonucleotide, or a combination thereof.

13. The inhibitor of claim 12, wherein the inhibitor is an siRNA comprising at least one nucleotide sequence selected from the group consisting of SEQ ID NOS: 3-45.

14. The pharmaceutical composition of claim 9, wherein the pharmaceutically acceptable carrier is a biologically compatible inert solvent.

15. The pharmaceutical composition of claim 9, wherein the pharmaceutically acceptable carrier is a virus, a liposome, an extracellular vesicle, or a polymer.

16. A method for treating a cancer in a subject in need thereof, comprising administering to the subject a pharmaceutically acceptable amount of the pharmaceutical composition of claim 8.

17. The method of claim 16, further comprising administering to the subject at least one additional anti-cancer drug.

18. The method of claim 16, wherein the cancer is a solid tumor or a disseminated cancer.

19. The method of claim 18, wherein the cancer cell is from a mammary gland cancer, a hepatocellular cancer, a pancreatic cancer, a colon cancer, a glioma, a lung cancer, a sarcoma, a bladder cancer, a leukemia, or a head and neck cancer.

20. A kit for targeting an X-box binding protein 1 variant (Xv1), comprising:
at least one Xv1 inhibitor;
at least one pharmaceutically acceptable carrier;
means for detecting the Xv1 protein or mRNA thereof; and
instructions for using the kit.

21. The kit of claim 20, wherein the Xv1 inhibitor is a nucleic acid, a protein, a peptide, or a small molecular weight compound, or a combination thereof.

22. The kit of claim 21, wherein the inhibitor is a nucleic acid designed from a nucleotide sequence of SEQ ID NO: 1 or SEQ ID NO: 2.

23. The kit of claim 20, wherein the Xv1 inhibitor is a small interfering RNA (siRNA), a Morpholino, a micro RNA (miRNA), a Piwi-interacting RNA (piRNA), a heterogeneous nuclear RNA (hnRNA), a small nuclear RNA (snRNA), a guide RNA (gRNA), a single guide RNA (sgRNA), a endoribonuclease-prepared small interfering RNA (esiRNA), a short hairpin RNA (shRNA), a clustered regularly interspaced short palindromic repeats (CRISPR)-based loss-of-function inhibitor, or an antisense oligonucleotide (for Xv1 knockdown or exon skipping), or a combination thereof.

24. The inhibitor of claim 23, wherein the inhibitor is an siRNA comprising at least one nucleotide sequence selected from the group consisting of SEQ ID NOS: 3-45.

25. The kit of claim 20, wherein the means for detecting the Xv1 protein is an antibody.

26. The kit of claim 20, wherein the means for detecting the Xv1 protein mRNA is an in situ hybridization probe.

27. The kit of claim 20, wherein the pharmaceutically acceptable carrier is a biologically compatible inert solvent.

28. The kit of claim 20, wherein the pharmaceutically acceptable carrier is a virus, a liposome, an extracellular vesicle, or a polymer.


SEQUENCE LISTING (83 sequences)

SEQ ID NO: 1 - DNA, 215 bp, Artificial Sequence
  cDNA sequence of X-box binding protein-1 variant XV1 first exon and C-terminus flanking sequence
  misc_feature 1..174: cDNA sequence of the first exon
  misc_feature 175..215: cDNA sequence of the C-terminus flanking sequence
ccggactgaccggatccgccacgctgggaacctagggcggcccagggctcttttctgtactttttaactctctcgttagagatgaccagagctggggatgcgggcacctgtcttccaggccctcttgctgtgtggccgcagactggtggttcagcctcttaactcggacatgaggaaactgaaaaacagagtagcagctcagactgccagagatc

SEQ ID NO: 2 - DNA, 224 bp, Artificial Sequence
  Genomic sequence of X-box binding protein-1 variant XV1 first exon and C-terminus flanking sequence
  misc_feature 1..174: genomic sequence of the first exon
  misc_feature 175..224: genomic sequence of the C-terminus flanking sequence
ccggactgaccggatccgccacgctgggaacctagggcggcccagggctcttttctgtactttttaactctctcgttagagatgaccagagctggggatgcgggcacctgtcttccaggccctcttgctgtgtggccgcagactggtggttcagcctcttaactcggacatgaggtcgaataatctgttttggtttactgctatttctggagaggcgcggagct

SEQ ID NO: 3 - RNA, 19 nt, Artificial Sequence
  siRNA sequence for inhibiting X-box binding protein-1 variant XV1 mRNA
ggauccgccacgcugggaa

SEQ ID NO: 4 - RNA, 19 nt, Artificial Sequence
  siRNA sequence for inhibiting X-box binding protein-1 variant XV1 mRNA
ccgccacgcugggaaccua

SEQ ID NO: 5 - RNA, 19 nt, Artificial Sequence
  siRNA sequence for inhibiting X-box binding protein-1 variant XV1 mRNA
cgccacgcugggaaccuag

SEQ ID NO: 6 - RNA, 19 nt, Artificial Sequence
  siRNA sequence for inhibiting X-box binding protein-1 variant XV1 mRNA
cggcccagggcucuuuucu

SEQ ID NO: 7 - RNA, 19 nt, Artificial Sequence
  siRNA sequence for inhibiting X-box binding protein-1 variant XV1 mRNA
gcccagggcucuuuucugu

SEQ ID NO: 8 - RNA, 19 nt, Artificial Sequence
  siRNA sequence for inhibiting X-box binding protein-1 variant XV1 mRNA
cccagggcucuuuucugua

SEQ ID NO: 9 - RNA, 19 nt, Artificial Sequence
  siRNA sequence for inhibiting X-box binding protein-1 variant XV1 mRNA
ccagggcucuuuucuguac

SEQ ID NO: 10 - RNA, 19 nt, Artificial Sequence
  siRNA sequence for inhibiting X-box binding protein-1 variant XV1 mRNA
agggcucuuuucuguacuu

SEQ ID NO: 11 - RNA, 19 nt, Artificial Sequence
  siRNA sequence for inhibiting X-box binding protein-1 variant XV1 mRNA
gggcucuuuucuguacuuu

SEQ ID NO: 12 - RNA, 19 nt, Artificial Sequence
  siRNA sequence for inhibiting X-box binding protein-1 variant XV1 mRNA
ggcucuuuucuguacuuuu

SEQ ID NO: 13 - RNA, 19 nt, Artificial Sequence
  siRNA sequence for inhibiting X-box binding protein-1 variant XV1 mRNA
gcucuuuucuguacuuuuu

SEQ ID NO: 14 - RNA, 19 nt, Artificial Sequence
  siRNA sequence for inhibiting X-box binding protein-1 variant XV1 mRNA
acuuuuuaacucucucguu

SEQ ID NO: 15 - RNA, 19 nt, Artificial Sequence
  siRNA sequence for inhibiting X-box binding protein-1 variant XV1 mRNA
cuuuuuaacucucucguua

SEQ ID NO: 16 - RNA, 19 nt, Artificial Sequence
  siRNA sequence for inhibiting X-box binding protein-1 variant XV1 mRNA
acucucucguuagagauga

SEQ ID NO: 17 - RNA, 19 nt, Artificial Sequence
  siRNA sequence for inhibiting X-box binding protein-1 variant XV1 mRNA
cucucucguuagagaugac

SEQ ID NO: 18 - RNA, 19 nt, Artificial Sequence
  siRNA sequence for inhibiting X-box binding protein-1 variant XV1 mRNA
cucucguuagagaugacca

SEQ ID NO: 19 - RNA, 19 nt, Artificial Sequence
  siRNA sequence for inhibiting X-box binding protein-1 variant XV1 mRNA
cucguuagagaugaccaga

SEQ ID NO: 20 - RNA, 19 nt, Artificial Sequence
  siRNA sequence for inhibiting X-box binding protein-1 variant XV1 mRNA
cguuagagaugaccagagc

SEQ ID NO: 21 - RNA, 19 nt, Artificial Sequence
  siRNA sequence for inhibiting X-box binding protein-1 variant XV1 mRNA
agaugaccagagcugggga

SEQ ID NO: 22 - RNA, 19 nt, Artificial Sequence
  siRNA sequence for inhibiting X-box binding protein-1 variant XV1 mRNA
ggaugcgggcaccugucuu

SEQ ID NO: 23 - RNA, 19 nt, Artificial Sequence
  siRNA sequence for inhibiting X-box binding protein-1 variant XV1 mRNA
ugcgggcaccugucuucca

SEQ ID NO: 24 - RNA, 19 nt, Artificial Sequence
  siRNA sequence for inhibiting X-box binding protein-1 variant XV1 mRNA
cugucuuccaggcccucuu

SEQ ID NO: 25 - RNA, 19 nt, Artificial Sequence
  siRNA sequence for inhibiting X-box binding protein-1 variant XV1 mRNA
cuugcuguguggccgcaga

SEQ ID NO: 26 - RNA, 19 nt, Artificial Sequence
  siRNA sequence for inhibiting X-box binding protein-1 variant XV1 mRNA
gccgcagacuggugguuca

SEQ ID NO: 27 - RNA, 19 nt, Artificial Sequence
  siRNA sequence for inhibiting X-box binding protein-1 variant XV1 mRNA
cagacuggugguucagccu

SEQ ID NO: 28 - RNA, 19 nt, Artificial Sequence
  siRNA sequence for inhibiting X-box binding protein-1 variant XV1 mRNA
gacuggugguucagccucu

SEQ ID NO: 29 - RNA, 19 nt, Artificial Sequence
  siRNA sequence for inhibiting X-box binding protein-1 variant XV1 mRNA
acuggugguucagccucuu

SEQ ID NO: 30 - RNA, 19 nt, Artificial Sequence
  siRNA sequence for inhibiting X-box binding protein-1 variant XV1 mRNA
uggugguucagccucuuaa

SEQ ID NO: 31 - RNA, 19 nt, Artificial Sequence
  siRNA sequence for inhibiting X-box binding protein-1 variant XV1 mRNA
ggugguucagccucuuaac

SEQ ID NO: 32 - RNA, 19 nt, Artificial Sequence
  siRNA sequence for inhibiting X-box binding protein-1 variant XV1 mRNA
gugguucagccucuuaacu

SEQ ID NO: 33 - RNA, 19 nt, Artificial Sequence
  siRNA sequence for inhibiting X-box binding protein-1 variant XV1 mRNA
gguucagccucuuaacucg

SEQ ID NO: 34 - RNA, 19 nt, Artificial Sequence
  siRNA sequence for inhibiting X-box binding protein-1 variant XV1 mRNA
agccucuuaacucggacau

SEQ ID NO: 35 - RNA, 19 nt, Artificial Sequence
  siRNA sequence for inhibiting X-box binding protein-1 variant XV1 mRNA
gccucuuaacucggacaug

SEQ ID NO: 36 - RNA, 19 nt, Artificial Sequence
  siRNA sequence for inhibiting X-box binding protein-1 variant XV1 mRNA
ccucuuaacucggacauga

SEQ ID NO: 37 - RNA, 19 nt, Artificial Sequence
  siRNA sequence for inhibiting X-box binding protein-1 variant XV1 mRNA
cucuuaacucggacaugag

SEQ ID NO: 38 - RNA, 19 nt, Artificial Sequence
  siRNA sequence for inhibiting X-box binding protein-1 variant XV1 mRNA
ucggacaugaggaaacuga

SEQ ID NO: 39 - RNA, 19 nt, Artificial Sequence
  siRNA sequence for inhibiting X-box binding protein-1 variant XV1 mRNA
cggacaugaggaaacugaa

SEQ ID NO: 40 - RNA, 19 nt, Artificial Sequence
  siRNA sequence for inhibiting X-box binding protein-1 variant XV1 mRNA
ggacaugaggaaacugaaa

SEQ ID NO: 41 - RNA, 19 nt, Artificial Sequence
  siRNA sequence for inhibiting X-box binding protein-1 variant XV1 mRNA
gacaugaggaaacugaaaa

SEQ ID NO: 42 - RNA, 19 nt, Artificial Sequence
  siRNA sequence for inhibiting X-box binding protein-1 variant XV1 mRNA
augaggaaacugaaaaaca

SEQ ID NO: 43 - RNA, 21 nt, Artificial Sequence
  siRNA sense sequence for X-box binding protein-1 variant XV1 mRNA
  misc_feature 20..21: n is deoxythymidine
ccucuuaacucggacaugann

SEQ ID NO: 44 - RNA, 21 nt, Artificial Sequence
  siRNA sense sequence for X-box binding protein-1 variant XV1 mRNA
  misc_feature 20..21: n is deoxythymidine
cucucguuagagaugaccann

SEQ ID NO: 45 - RNA, 21 nt, Artificial Sequence
  siRNA sense sequence for X-box binding protein-1 mRNA
  misc_feature 20..21: n is deoxythymidine
gaccccuaaaguucugcuunn

SEQ ID NO: 46 - RNA, 21 nt, Artificial Sequence
  siRNA sense sequence for Tubulin Tyrosine Ligase Like 6 (TTLL6) mRNA
  misc_feature 20..21: n is deoxythymidine
gaaacguaugagaaggaaann

SEQ ID NO: 47 - RNA, 21 nt, Artificial Sequence
  siRNA sense sequence for Tubulin Tyrosine Ligase Like 6 (TTLL6) mRNA
  misc_feature 20..21: n is deoxythymidine
gcuuugcgacgaccucuuann

SEQ ID NO: 48 - RNA, 21 nt, Artificial Sequence
  siRNA sense sequence for Tubulin Tyrosine Ligase Like 6 (TTLL6) mRNA
  misc_feature 20..21: n is deoxythymidine
gggauuaaugaaacugccann

SEQ ID NO: 49 - protein, 18 aa, Artificial Sequence
  Peptide sequence common to all X-box binding protein-1 Proteins and X-box binding protein-1 variant proteins
  SITE 18: Cysteine is added at position 18 and is conjugated to keyhole limpet hemocyanin
RQRLGMDALVAEEEAEAC

SEQ ID NO: 50 - DNA, 17 bp, Artificial Sequence
  Primer sequence for X-box binding protein-1
gaggagaaggcgctgag

SEQ ID NO: 51 - DNA, 19 bp, Artificial Sequence
  Primer sequence for X-box binding protein-1
cactggcctcacttcattc

SEQ ID NO: 52 - DNA, 21 bp, Artificial Sequence
  Primer sequence for X-box binding protein-1 variant XV1
ggtggttcagcctcttaactc

SEQ ID NO: 53 - DNA, 19 bp, Artificial Sequence
  Primer sequence for X-box binding protein-1 variant XV1
cactggcctcacttcattc

SEQ ID NO: 54 - DNA, 21 bp, Artificial Sequence
  Primer sequence for X-box binding protein-1 spliced variant XV1s
ggtggttcagcctcttaactc

SEQ ID NO: 55 - DNA, 19 bp, Artificial Sequence
  Primer sequence for X-box binding protein-1 spliced variant XV1s
ctgcacctgctgcgtactc

SEQ ID NO: 56 - DNA, 21 bp, Artificial Sequence
  Primer sequence for X-box binding protein-1 unspliced variant XV1s
ggtggttcagcctcttaactc

SEQ ID NO: 57 - DNA, 19 bp, Artificial Sequence
  Primer sequence for X-box binding protein-1 unspliced variant XV1s
gaggtgcacgtagtctgag

SEQ ID NO: 58 - DNA, 18 bp, Artificial Sequence
  Primer sequence for beta-actin
caccaactgggacgacat

SEQ ID NO: 59 - DNA, 18 bp, Artificial Sequence
  Primer sequence for beta-actin
acagcctggatagcaacg

SEQ ID NO: 60 - DNA, 19 bp, Artificial Sequence
  Primer sequence for Tubulin Tyrosine Ligase Like 6
gcgaagcccttcagttctc

SEQ ID NO: 61 - DNA, 22 bp, Artificial Sequence
  Primer sequence for Tubulin Tyrosine Ligase Like 6
gctcctctcacatcctttttgg

SEQ ID NO: 62 - DNA, 18 bp, Artificial Sequence
  Primer sequence for X-box binding protein-1 variant XV1
ggtgcaggcccagttgtc

SEQ ID NO: 63 - DNA, 19 bp, Artificial Sequence
  Primer sequence for X-box binding protein-1 variant XV1
tgctgcggactcagcagac

SEQ ID NO: 64 - DNA, 23 bp, Artificial Sequence
  Primer sequence for X-box binding protein-1 variant XV1
cgggtctgcagaatccgcagcac

SEQ ID NO: 65 - DNA, 23 bp, Artificial Sequence
  Primer sequence for X-box binding protein-1 variant XV1
gtgctgcggattctgcagacccg

SEQ ID NO: 66 - DNA, 27 bp, Artificial Sequence
  Primer sequence for site-directed mutagenesis to create sirna resistant XV1
cctcttaactctgatatgaggaaactg

SEQ ID NO: 67 - DNA, 27 bp, Artificial Sequence
  Primer sequence for site-directed mutagenesis to create sirna resistant XV1
cagtttcctcatatcagagttaagagg

SEQ ID NO: 68 - DNA, 32 bp, Artificial Sequence
  Primer sequence for X-box binding protein-1 unspliced variant XV1u
  modified_base 32: Hemagglutinin (HA) tag at position 32
ctttgctagccaccatgcgggcacctgtcttc

SEQ ID NO: 69 - DNA, 62 bp, Artificial Sequence
  Primer sequence for X-box binding protein-1 unspliced variant XV1u
  modified_base 62: Hemagglutinin (HA) tag at position 62
gtttgcggccgcttaagcgtaatctggaacatcgtatgggtagttcattaatggcttccagc

SEQ ID NO: 70 - DNA, 32 bp, Artificial Sequence; identical to SEQ ID NO: 68
  Primer sequence for X-box binding protein-1 variant XV1

SEQ ID NO: 71 - DNA, 31 bp, Artificial Sequence
  Primer sequence for X-box binding protein-1 variant XV1
gaaaggatccgttagacactaatcagctggg

SEQ ID NO: 72 - DNA, 39 bp, Artificial Sequence
  Primer sequence for Tubulin Tyrosine Ligase Like 6
cttcactagtgcagccaatgggagcgttactccttcatc

SEQ ID NO: 73 - DNA, 38 bp, Artificial Sequence
  Primer sequence for Tubulin Tyrosine Ligase Like 6
gacttgcggccgcttagctcctctcacatcctttttgg

SEQ ID NO: 74 - DNA, 28 bp, Artificial Sequence
  Primer sequence for X-box binding protein-1
gttcactagtgcgtagtctggagctatg

SEQ ID NO: 75 - DNA, 30 bp, Artificial Sequence
  Primer sequence for X-box binding protein-1
ctttggatccagacaggcttctctgctatc

SEQ ID NO: 76 - DNA, 171 bp, Artificial Sequence
  Unique sequence of X-box binding protein-1 alternate spliced variant 4 (XBP1AS4)
ccggactgaccggatccgccacgctgggaacctagggcggcccagggctcttttctgtactttttaactctctcgttagagatgaccagagctggggatgcgggcacctgtcttccaggccctcttgctgtgtggccgcagactggtggttcagcctcttaactcggacat

SEQ ID NO: 77 - DNA, 1690 bp, Artificial Sequence
  Sequence of full length X-box binding protein-1 alternate spliced variant 4 (XBP1AS4)
ccggactgaccggatccgccacgctgggaacctagggcggcccagggctcttttctgtactttttaactctctcgttagagatgaccagagctggggatgcgggcacctgtcttccaggccctcttgctgtgtggccgcagactggtggttcagcctcttaactcggacatgaggaaactgaaaaacagagtagcagctcagactgccagagatcgaaagaaggctcgaatgagtgagctggaacagcaagtggtagatttagaagaagagaaccaaaaacttttgctagaaaatcagcttttacgagagaaaactcatggccttgtagttgagaaccaggagttaagacagcgcttggggatggatgccctggttgctgaagaggaggcggaagccaaggggaatgaagtgaggccagtggccgggtctgctgagtccgcagcactcagactacgtgcacctctgcagcaggtgcaggcccagttgtcacccctccagaacatctccccatggattctggcggtattgactcttcagattcagagtctgatatcctgttgggcattctggacaacttggacccagtcatgttcttcaaatgcccttccccagagcctgccagcctggaggagctcccagaggtctacccagaaggacccagttccttaccagcctccctttctctgtcagtggggacgtcatcagccaagctggaagccattaatgaactaattcgttttgaccacatatataccaagcccctagtcttagagataccctctgagacagagagccaagctaatgtggtagtgaaaatcgaggaagcacctctcagcccctcagagaatgatcaccctgaattcattgtctcagtgaaggaagaacctgtagaagatgacctcgttccggagctgggtatctcaaatctgctttcatccagccactgcccaaagccatcttcctgcctactggatgcttacagtgactgtggatacgggggttccctttccccattcagtgacatgtcctctctgcttggtgtaaaccattcttgggaggacacttttgccaatgaactctttccccagctgattagtgtctaaggaatgatccaatactgttgcccttttccttgactattacactgcctggaggatagcagagaagcctgtctgtacttcattcaaaaagccaaaatagagagtatacagtcctagagaattcctctatttgttcagatctcatagatgacccccaggtattgtcttttgacatccagcagtccaaggtattgagacatattactggaagtaagaaatattactataattgagaactacagcttttaagattgtacttttatcttaaaagggtggtagttttccctaaaatacttattatgtaagggtcattagacaaatgtcttgaagtagacatggaatttatgaatggttctttatcatttctcttccccctttttggcatcctggcttgcctccagttttaggtcctttagtttgcttctgtaagcaacgggaacacctgctgagggggctctttccctcatgtatacttcaagtaagatcaagaatcttttgtgaaattatagaaatttactatgtaaatgcttgatggaattttttcctgctagtgtagcttctgaaaggtgctttctccatttatttaaaa

SEQ ID NO: 78 - DNA, 200 bp, Artificial Sequence
  Sequence for X-box binding protein-1 alternate spliced variant 4 (XBP1AS4)
ccggactgaccggatccgccacgctgggaacctagggcggcccagggctcttttctgtactttttaactctctcgttagagatgaccagagctggggatgcgggcacctgtcttccaggccctcttgctgtgtggccgcagactggtggttcagcctcttaactcggacatgaggaaactgaaaaacagagtagcagctc

SEQ ID NO: 79 - protein, 211 aa, Artificial Sequence
  Unspliced form of X-box binding protein-1 alternate spliced variant 4 (XBP1AS4)
MRAPVFQALLLCGRRLVVQPLNSDMRKLKNRVAAQTARDRKKARMSELEQQVVDLEEENQKLLLENQLLREKTHGLVVENQELRQRLGMDALVAEEEAEAKGNEVRPVAGSAESAALRLRAPLQQVQAQLSPLQNISPWILAVLTLQIQSLISCWAFWTTWTQSCSSNALPQSLPAWRSSQRSTQKDPVPYQPPFLCQWGRHQPSWKPLMN

SEQ ID NO: 80 - protein, 326 aa, Artificial Sequence
  Spliced form of X-box binding protein-1 alternate spliced variant 4 (XBP1AS4)
MRAPVFQALLLCGRRLVVQPLNSDMRKLKNRVAAQTARDRKKARMSELEQQVVDLEEENQKLLLENQLLREKTHGLVVENQELRQRLGMDALVAEEEAEAKGNEVRPVAGSAESAAGAGPVVTPPEHLPMDSGGIDSSDSESDILLGILDNLDPVMFFKCPSPEPASLEELPEVYPEGPSSLPASLSLSVGTSSAKLEAINELIRFDHIYTKPLVLEIPSETESQANVVVKIEEAPLSPSENDHPEFIVSVKEEPVEDDLVPELGISNLLSSSHCPKPSSCLLDAYSDCGYGGSLSPFSDMSSLLGVNHSWEDTFANELFPQLISV

SEQ ID NO: 81 - protein, 25 aa, Artificial Sequence
  N-terminal aminoacids of X-box binding protein-1 alternate spliced variant 4 (XBP1AS4)
MRAPVFQALLLCGRRLVVQPLNSDM

SEQ ID NO: 82 - protein, 36 aa, Artificial Sequence
  N-terminal aminoacids of X-box binding protein-1 alternate spliced variant 4 (XBP1AS4)
MRAPVFQALLLCGRRLVVQPLNSDMRKLKNRVAAQT

SEQ ID NO: 83 - protein, 75 aa, Artificial Sequence
  N-terminal amino acids of X-box binding protein-1 alternate spliced variant 4 (XBP1AS4)
MVVVAAAPNPADGTPKVLLLSGQPASAAGAPAGQALPLMVPAQRGASPEAASGGLPQARKRQRLTHLSPEEKALR